(19) United States
(12) Patent Application Publication
(10) Pub. No.: US 2014/0100364 A1
(43) Pub. Date: Apr. 10, 2014

(54) Compounds Useful For Making HCV Protease Inhibitors

(71) Applicant: AbbVie Inc., North Chicago, IL (US)

(72) Inventors: Kenneth M. Engstrom, Mundelein, IL (US); Yanqun Zhao, Lake Villa, IL (US)

(21) Appl. No.: 14/047,440

(22) Filed: Oct. 7, 2013

Related U.S. Application Data
(60) Provisional application No. 61/711,001, filed on Oct. 8, 2012.

(51) Int. Cl. C07D 498/16; C07D 417/14; C07D 487/04; C07D 498/06

(52) U.S. Cl. CPC C07D 498/16; C07D 498/06; C07D 417/14; C07D 487/04
USPC 540/456; 540/460

(57) ABSTRACT

Compounds useful for making HCV protease inhibitors are described. Methods of using these compounds to make HCV protease inhibitors are also provided.

This application claims priority to U.S. Provisional Application No. 61/711,001 filed Oct. 8, 2012, which is incorporated by reference in its entirety.

### FIELD

The present invention relates to compounds useful for making HCV protease inhibitors and methods of using the same to make HCV protease inhibitors.

### BACKGROUND

HCV is the principal cause of non-A, non-B hepatitis and is an increasingly severe public health problem both in the developed and developing world. It is estimated that the virus infects over 200 million people worldwide, surpassing the number of individuals infected with the human immunodeficiency virus (HIV) by nearly five fold. HCV infected patients are at an elevated risk of developing cirrhosis of the liver, subsequent hepatocellular carcinoma and terminal liver disease. HCV is the most prevalent cause of hepatocellular cancer in the western world.

The HCV protease mediates the cleavage of the HCV polyprotein to release the functional proteins that are essential for viral propagation. The inhibition of the HCV protease activity is expected to block HCV replication in infected host cells. Numerous HCV protease inhibitors have been identified. Non-limiting examples of HCV protease inhibitors are described in U.S. Patent Application Pub. Nos. 20030181363, 20030224977, 2003181363, 2003224977, 2004002448, 20040180815, 20040229776, 20040248779, 20040266668, 2004038872, 2004229777, 2004266668, 20050075279, 20050080005, 20050090432, 20050153877, 20050209135, 20050267018, 2005080005, 2005192212, 20060009667, 20060063915, 20060063916, 20060089300, 20060122123, 20060122123, 20060205638, 20060257980, 2006257980, 20070060510, 20070237818, 20070281885, 20070299078, 2007060510, 2007099825, 2007237818, 2007258947, 2007281884, 2007281885, 2007299078, 20080008681, 20080039375, 20080039470, 20080152622, 20080181868, 20080242835, 20080267917, 20080269228, 20080269502, 20080279821, 2008039375, 2008039470, 20090005387, 20090035271, 20090130059, 20090130059, 20090148407, 20090163706, 20090163706, 20090186869, 20090202480, 20090257978, 20090285773, 20090286814, 20090297472, 20090304629, 20090306085, 20090326194, 20100015092, 20100036116, 20100196321, 20100260710, 20100286185, 20110059047, 20110123496, 20110135604, 20110178107, 20110183895, and 20120095211, and U.S. Pat. Nos. 6,608,027, 6,867,185, 6,867,185, 7,119,072, 7,157,424, 7,173,004, 7,176,208, 7,189,844, 7,368,452, 7,375,218, 7,504,378, 7,566,719, 7,763,584, 7,772,183, 7,829,665, and 7,910,728, as well as WO2007014919, WO2007014926, WO2008046860, WO2008057995, WO2008095058, WO2009139792, WO2010122087, and WO2011034518. Many of these protease inhibitors have macrocyclic structures as depicted in Formula A or Formula B described below.

### DETAILED DESCRIPTION

In one aspect, the present invention features compounds of Formula I and salts thereof,

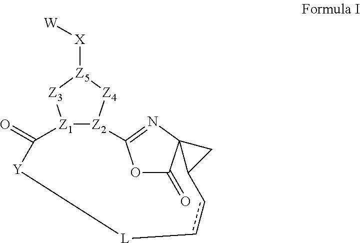

wherein:

W is optionally substituted carbocycle or heterocycle;

X is absent, —O—, —S—, —N(R_{N})—, —OC(O)—, —C(O)—, —C(O)O—, —N(R_{N})C(O)—, C(O)N(R_{N})—, —S(O)— or —S(O)₂—; or X is optionally substituted C₁-C₆alkylene, C₂-C₆alkenylene or C₂-C₆alkynylene, each of said C₁-C₆alkylene, C₂-C₆alkenylene or C₂-C₆alkynylene containing 0, 1, 2, or 3 heteroatoms independently selected from O, S or N;

Z₁, Z₂ and Z₅ are each independently —C(R_{C})— or —N—;

Z₃ and Z₄, together with Z₁, Z₂ and Z₅, form an optionally substituted 5-membered, 6-membered or 7-membered carbocycle or heterocycle;

Y is C(R_{A}R_{B}) or N(R_{A});

L is optional substituted C₃-C₈alkylene, C₃-C₈alkenylene or C₃-C₈alkynylene, each said C₃-C₈alkylene, C₃-C₈alkenylene or C₃-C₈alkynylene containing 0, 1, 2, or 3 heteroatoms independently selected from O, S or N;

R_{N} is independently selected at each occurrence from hydrogen; or optionally substituted C₁-C₆alkyl, C₂-C₆alkenyl or C₂-C₆alkynyl; or optionally substituted 3- to 6-membered carbocycle or heterocycle;

R_{C} is independently selected at each occurrence from hydrogen, halogen, hydroxy, mercapto, amino, carboxy, nitro, oxo, phosphonoxy, phosphono, thioxo, formyl or cyano; or optionally substituted C₁-C₆alkyl, C₂-C₆alkenyl or C₂-C₆alkynyl; or optionally substituted 3- to 6-membered carbocycle or heterocycle;

each R_{A} and R_{B} is independently selected at each occurrence from hydrogen, halogen, hydroxy, mercapto, amino, carboxy, nitro, phosphonoxy, phosphono, formyl, cyano, or -L₁-R_{D};

L₁ is independently selected at each occurrence from absent; or optionally substituted C₁-C₆alkylene, C₂-C₆alkenylene or C₂-C₆alkynylene, each of said C₁-C₆alkylene, C₂-C₆alkenylene or C₂-C₆alkynylene containing 0, 1, 2, or 3 heteroatoms independently selected from O, S or N;

R_{D} is independently selected at each occurrence from —O—R_{S}, —S—R_{S}, —C(O)R_{S}, —OC(O)R_{S}, —C(O)OR_{S}, —N(R_{N})R_{S}, —S(O)R_{S}, —SO₂R_{S}, —C(O)N(R_{N})R_{S}, N(R_{N})C(O)R_{S}, —N(R_{N})C(O)N(R_{N})R_{S}, —N(R_{N})SO₂R_{S}, —SO₂N(R_{N})R_{S}, —N(R_{N})SO₂N(R_{N})R_{S}, N(R_{N})S(O)N(R_{N})R_{S}, —OS(O)—R_{S}, —OS(O)₂—R_{S}, —S(O)₂OR_{S}, —S(O)OR_{S}, —OC(O)OR_{S}, —N(R_{N})C(O)OR_{S}, —OC(O)N(R_{N})R_{S}, —N(R_{N})S(O)—R_{S}, —S(O)N(R_{N})R_{S}, —P(O)(OR_{S})₂, or —C(O)N(R_{N})C(O)—R_{S}; or optionally substituted C₁-C₆alkyl, C₂-C₆alkenyl or C₂-C₆alkynyl; or optionally substituted carbocycle or heterocycle;

R_{S} is independently selected at each occurrence from hydrogen; optionally substituted C₁-C₆alkyl, C₂-C₆alkenyl or C₂-C₆alkynyl; or optionally substituted carbocycle or heterocycle; and

[symbol] denotes optionally substituted —CH₂—CH₂— or optionally substituted —CH═CH—.

W preferably is an optionally substituted 9-, 10- or 11-membered carbocycle or heterocycle which comprises two fused rings. Non-limiting examples of such carbocycles or heterocycles include naphthyl, indanyl, 1,2,3,4-tetrahydro-naphthyl, indenyl, isoindenyl, decalinyl, norpinanyl, naphthyridinyl (including [1,8] naphthyridinyl, and [1,6] naphthyridinyl), thiazolpyrimidinyl, thienopyrimidinyl, pyrimidopyrimidinyl, pyridopyrimidinyl, pyrazolopyrimidinyl, indolizinyl, pyrindinyl, pyranopyrrolyl, 4H-quinolizinyl, purinyl, pyridopyridinyl (including pyrido[3,4-b]-pyridinyl, pyrido[3,2-b]-pyridinyl, and pyrido[4,3-b]-pyridinyl), pyridopyrimidine, and pteridinyl. Other non-limiting examples of fused-ring heterocycles include benzo-fused heterocyclyls, such as indolyl, isoindolyl, indoleninyl (also known as “pseudoindolyl”), isoindazolyl (also known as “benzpyrazolyl” or indazolyl), benzazinyl (including quinolinyl (also known as “1-benzazinyl”) and isoquinolinyl (also known as “2-benzazinyl”)), benzimidazolyl, phthalazinyl, quinoxalinyl, benzodiazinyl (including cinnolinyl (also known as “1,2-benzodiazinyl”) and quinazolinyl (also known as “1,3-benzodiazinyl”)), benzopyranyl (including “chromenyl” and “isochromenyl”), benzothiopyranyl (also known as “thiochromenyl”), benzoxazolyl, indoxazinyl (also known as “benzisoxazolyl”), anthranilyl, benzodioxolyl, benzodioxanyl, benzoxadiazolyl, benzofuranyl (also known as “coumaronyl”), isobenzofuranyl, benzothienyl (also known as “benzothiophenyl”, “thionaphthenyl”, and “benzothiofuranyl”), isobenzothienyl (also known as “isobenzothiophenyl”, “isothionaphthenyl”, and “isobenzothiofuranyl”), benzothiazolyl, 4,5,6,7-tetrahydrobenzo[d]thiazolyl, benzothiadiazolyl, benzimidazolyl, benzotriazolyl, benzoxazinyl (including 1,3,2-benzoxazinyl, 1,4,2-benzoxazinyl, 2,3,1-benzoxazinyl, and 3,1,4-benzoxazinyl), benzisoxazinyl (including 1,2-benzisoxazinyl and 1,4-benzisoxazinyl), and tetrahydroisoquinolinyl.

Also preferably, W is an optionally substituted 12-, 13-, 14-, 15-, or 16-membered carbocycle or heterocycle which comprises three fused rings. Highly preferably, W is an optionally substituted 12-, 13-, 14-, 15-, or 16-membered carbocycle or heterocycle which comprises three fused

rings, provided that W is not

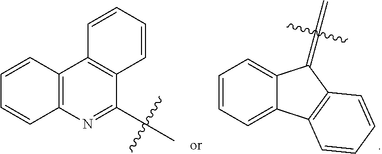 [SMILES: *c1nc2ccccc2c2ccccc12 ; *=C1c2ccccc2-c2ccccc21]

Non-limiting examples of such carbocycles or heterocycles include the bicyclic ring systems described above further fused with another 5- or 6-membered monocyclic carbocycle or heterocycle.

Z₁, Z₂, Z₃, Z₄ and Z₅ preferably form a 5- or 6-membered carbocycle or heterocycle. More preferably, Z₁, Z₂, Z₃, Z₄ and Z₅ form a 5-membered carbocycle or heterocycle. Highly preferably, Z₁, Z₂, Z₃, Z₄ and Z₅ form a 5-membered, saturated carbocycle or heterocycle. More preferably, Z₁ is N, Z₂ is C(R_{C}), Z₃ is C(R_{C})₂, Z₄ is C(R_{C})₂, and Z₅ is C(R_{C}). Most preferably, Z₁ is N, Z₂ is C(H), Z₃ is CH₂, Z₄ is CH₂, and Z₅ is CH.

X preferably is —O—, —OC(O)— or —C(O)O—. Highly preferably, X is —OC(O)— or —C(O)O—. More preferably, X is —OC(O)—, wherein —O— is directly linked to Z₅, and —C(O)— is directly linked to W. Also more preferably, X is —O—.

L preferably is an optionally substituted C₄-C₆alkylene. L preferably is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

More preferably, L is an optionally substituted, straight C₄-C₆alkylene. L preferably is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

Highly preferably, L is an optionally substituted straight C₅alkylene. L preferably is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

Most preferably, L is —CH₂—CH₂—CH₂—CH₂—CH₂—.

Y preferably is —CH(N(R_{N})C(O)R_{S})—, —CH(N(R_{N})C(O)N(R_{N})R_{S})—, or CH(N(R_{N})C(O)OR_{S})—. More preferably, Y is —CH(N(R_{N})C(O)R_{S}′)—, wherein R_{S}′ is (i) hydrogen; (ii) C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle; or (iii) 3- to 6-membered carbocycle or heterocycle, and wherein each 3- to 6-membered carbocycle or heterocycle in R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl. Highly preferably, Y is —CH(N(R_{N})C(O)R_{S}′)—, wherein R_{S}′ is 3- to 6-membered carbocycle or heterocycle, which is optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

Also preferably, Y is —CH(N(R_{N})C(O)OR_{S}′)—, wherein R_{S}′ is (i) hydrogen; (ii) C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle; or (iii) 3- to 6-membered carbocycle or heterocycle, and wherein each 3- to 6-membered carbocycle or heterocycle in R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl. Highly preferably, Y is —CH(N(R_{N})C(O)OR_{S}′)—, wherein R_{S}′ is C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle.
[symbol] preferably is —CH═CH—.

Also preferably, [symbol] is —CH₂—CH₂— which is optionally substituted with one or more halogens. More preferably, [symbol] is a —C(R₁R₂)—C(R₃R₄)—, wherein R₁ and R₃ are hydrogen, R₂ and R₄ are halogen (e.g., F); or [symbol] is a —C(R₁R₂)—C(R₃R₄)—, wherein R₁, R₂, R₃ and R₄ are halogen (e.g., F).

In one embodiment, W is a 9-, 10- or 11-membered fused bicycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —O—; Z₁ is N, Z₂ is C(R_{C}), Z₃ is C(R_{C})₂, Z₄ is C(R_{C})₂, and Z₅ is C(R_{C}); L is a straight C₄-C₆alkylene which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; Y is —CH(N(R_{N})C(O)R_{S}′)— or —CH(N(R_{N})C(O)OR_{S}′)—; and [symbol] is —CH═CH— or —C(R₁R₂)—C(R₃R₄)—, wherein R₁, R₂, R₃ and R₄ are each independently hydrogen or halogen. Said 9-, 10- or 11-membered fused bicycle can be either carbocycle or heterocycle. Preferably, said 9-, 10- or 11-membered fused bicycle is selected from quinoline, isoquinoline, quinoxaline or isoindoline.

In another embodiment, W is a 9-, 10- or 11-membered fused bicycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —O—; Z₁ is N, Z₂ is C(H), Z₃ is CH₂, Z₄ is CH₂, and Z₅ is CH; L is a straight C₄-C₆alkylene which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; Y is —CH(N(R_{N})C(O)R_{S}′)— or —CH(N(R_{N})C(O)OR_{S}′)—; and [symbol] is —CH═CH— or —C(R₁R₂)—C(R₃R₄)—, wherein R₁, R₂, R₃ and R₄ are each independently hydrogen or halogen. Said 9-, 10- or 11-membered fused bicycle can be either carbocycle or heterocycle. Preferably, said 9-, 10- or 11-membered fused bicycle is selected from quinoline, isoquinoline, quinoxaline or isoindoline.

In another embodiment, W is a 9-, 10- or 11-membered fused bicycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —O—; Z₁ is N, Z₂ is C(H), Z₃ is CH₂, Z₄ is CH₂, and Z₅ is CH; L is a straight C₅alkylene, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; Y is —CH(N(R_{N})C(O)R_{S}′)— or —CH(N(R_{N})C(O)OR_{S}′)—; and [symbol] is —CH═CH— or —C(R₁R₂)—C(R₃R₄)—, wherein R₁, R₂, R₃ and R₄ are each independently hydrogen or halogen. Said 9-, 10- or 11-membered fused bicycle can be either carbocycle or heterocycle. Preferably, said 9-, 10- or 11-membered fused bicycle is selected from quinoline, isoquinoline, quinoxaline or isoindoline.

In another embodiment, W is a 9-, 10- or 11-membered fused bicycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —O—; Z₁ is N, Z₂ is C(H), Z₃ is CH₂, Z₄ is CH₂, and Z₅ is CH; L is —CH₂—CH₂—CH₂—CH₂—CH₂—; Y is —CH(N(R_{N})C(O)R_{S}′)— or —CH(N(R_{N})C(O)OR_{S}′)—; and [symbol] is —CH═CH— or —C(R₁R₂)—C(R₃R₄)—, wherein R₁, R₂, R₃ and R₄ are each independently hydrogen or halogen. Said 9-, 10- or 11-membered fused bicycle can be either carbocycle or heterocycle. Preferably, said 9-, 10- or 11-membered fused bicycle is selected from quinoline, isoquinoline, quinoxaline or isoindoline.

In another embodiment, W is a 9-, 10- or 11-membered fused bicycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —O—; Z₁ is N, Z₂ is C(H), Z₃ is CH₂, Z₄ is CH₂, and Z₅ is CH; L is —CH₂—CH₂—CH₂—CH₂—CH₂—; Y is —CH(N(R_{N})C(O)R_{S}′)— or —CH(N(R_{N})C(O)OR_{S}′)—; and [symbol] is —CH═CH—. Said 9-, 10- or 11-membered fused bicycle can be either carbocycle or heterocycle. Preferably, said 9-, 10- or 11-membered fused bicycle is selected from quinoline, isoquinoline, quinoxaline or isoindoline. R_{S}′ preferably is (i) C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle; or (ii) 3- to 6-membered carbocycle or heterocycle, and wherein each 3- to 6-membered carbocycle or heterocycle in R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

In another embodiment, W is a 9-, 10- or 11-membered fused bicycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —O—; Z₁ is N, Z₂ is C(H), Z₃ is CH₂, Z₄ is CH₂, and Z₅ is CH; L is —CH₂—CH₂—CH₂—CH₂—CH₂—; Y is —CH(N(R_{N})C(O)R_{S}′)— or —CH(N(R_{N})C(O)OR_{S}′)—; and [symbol] is —C(R₁R₂)—C(R₃R₄)—, wherein R₁, R₂, R₃ and R₄ are each independently hydrogen or halogen. Said 9-, 10- or 11-membered fused bicycle can be either carbocycle or heterocycle. Preferably, said 9-, 10- or 11-membered fused bicycle is selected from quinoline, isoquinoline, quinoxaline or isoindoline. R_{S}′ preferably is (i) C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle; or (ii) 3- to 6-membered carbocycle or heterocycle, and wherein each 3- to 6-membered carbocycle or heterocycle in R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl. Preferably, R₁ and R₃ are hydrogen, and R₂ and R₄ are halogen (e.g., F). Also preferably, R₁, R₂, R₃ and R₄ are halogen (e.g., F).

In another embodiment, W is a 9-, 10- or 11-membered fused bicycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —O—; Z₁ is N, Z₂ is C(H), Z₃ is CH₂, Z₄ is CH₂, and Z₅ is CH; L is —CH₂—CH₂—CH₂—CH₂—CH₂—; Y is —CH(N(R_{N})C(O)R_{S}′)—; and [symbol] is —CH═CH— or —C(R₁R₂)—C(R₃R₄)—, wherein R₁, R₂, R₃ and R₄ are each independently hydrogen or halogen. R_{S}′ is (i) hydrogen; (ii) C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle; or (iii) 3- to 6-membered carbocycle or heterocycle, and wherein each 3- to 6-membered carbocycle or heterocycle in R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl. Preferably, R_{S}′ is 5- to 6-membered carbocycle or heterocycle which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl. Said 9-, 10- or 11-membered fused bicycle can be either carbocycle or heterocycle. Preferably, said 9-, 10- or 11-membered fused bicycle is selected from quinoline, isoquinoline, quinoxaline or isoindoline.

In another embodiment, W is a 12-, 13-, 14-, 15-, or 16-membered fused tricycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —O—; Z₁ is N, Z₂ is C(R_{C}), Z₃ is C(R_{C})₂, Z₄ is C(R_{C})₂, and Z₅ is C(R_{C}); L is a straight C₄-C₆alkylene which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; Y is —CH(N(R_{N})C(O)R_{S}′)— or —CH(N(R_{N})C(O)OR_{S}′)—; and [symbol] is —CH═CH— or —C(R₁R₂)—C(R₃R₄)—, wherein R₁, R₂, R₃ and R₄ are each independently hydrogen or halogen. Said 12-, 13-, 14-, 15-, or 16-membered fused tricycle can be either carbocycle or heterocycle. Preferably, said 12-, 13-, 14-, 15-, or 16-membered fused tricycle is not

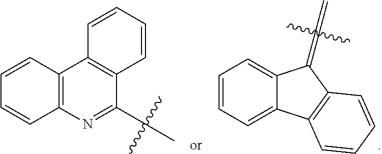 [SMILES: *c1nc2ccccc2c2ccccc12 ; *=C1c2ccccc2-c2ccccc21]

In another embodiment, W is a 12-, 13-, 14-, 15-, or 16-membered fused tricycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —O—; Z₁ is N, Z₂ is C(H), Z₃ is CH₂, Z₄ is CH₂, and Z₅ is CH; L is a straight C₄-C₆alkylene which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; Y is —CH(N(R_{N})C(O)R_{S}′)— or —CH(N(R_{N})C(O)OR_{S}′)—; and [symbol] is —CH═CH— or —C(R₁R₂)—C(R₃R₄)—, wherein R₁, R₂, R₃ and R₄ are each independently hydrogen or halogen. Said 12-, 13-, 14-, 15-, or 16-membered fused tricycle can be either carbocycle or heterocycle. Preferably, said 12-, 13-, 14-, 15-, or 16-membered fused tricycle is not

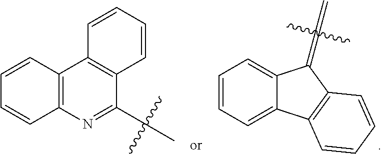 [SMILES: *c1nc2ccccc2c2ccccc12 ; *=C1c2ccccc2-c2ccccc21]

In another embodiment, W is a 12-, 13-, 14-, 15-, or 16-membered fused tricycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —O—; Z₁ is N, Z₂ is C(H), Z₃ is CH₂, Z₄ is CH₂, and Z₅ is CH; L is a straight C₅alkylene, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; Y is —CH(N(R_{N})C(O)R_{S}′)— or —CH(N(R_{N})C(O)OR_{S}′)—; and [symbol] is —CH═CH— or —C(R₁R₂)—C(R₃R₄)—, wherein R₁, R₂, R₃ and R₄ are each independently hydrogen or halogen. Said 12-, 13-, 14-, 15-, or 16-membered fused tricycle can be either carbocycle or heterocycle. Preferably, said 12-, 13-, 14-, 15-, or 16-membered fused tricycle is not

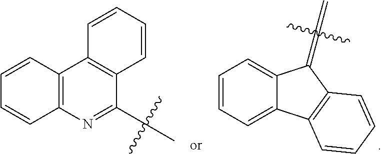 [SMILES: *c1nc2ccccc2c2ccccc12 ; *=C1c2ccccc2-c2ccccc21]

In another embodiment, W is a 12-, 13-, 14-, 15-, or 16-membered fused tricycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —O—; Z₁ is N, Z₂ is C(H), Z₃ is CH₂, Z₄ is CH₂, and Z₅ is CH; L is —CH₂—CH₂—CH₂—CH₂—CH₂—; Y is —CH(N(R_{N})C(O)R_{S}′)— or —CH(N(R_{N})C(O)OR_{S}′)—; and [symbol] is —CH═CH— or —C(R₁R₂)—C(R₃R₄)—, wherein R₁, R₂, R₃ and R₄ are each independently hydrogen or halogen. Said 12-, 13-, 14-, 15-, or 16-membered fused tricycle can be either carbocycle or heterocycle. Preferably, said 12-, 13-, 14-, 15-, or 16-membered fused tricycle is not

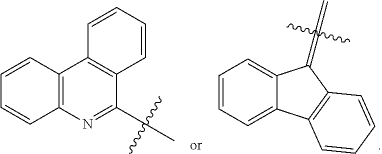 [SMILES: *c1nc2ccccc2c2ccccc12 ; *=C1c2ccccc2-c2ccccc21]

In another embodiment, W is a 12-, 13-, 14-, 15-, or 16-membered fused tricycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —O—; Z₁ is N, Z₂ is C(H), Z₃ is CH₂, Z₄ is CH₂, and Z₅ is CH; L is —CH₂—CH₂—CH₂—CH₂—CH₂—; Y is —CH(N(R_{N})C(O)R_{S}′)— or —CH(N(R_{N})C(O)OR_{S}′)—; and [symbol] is —CH═CH—. Said 12-, 13-, 14-, 15-, or 16-membered fused tricycle can be either carbocycle or heterocycle. Preferably, said 12-, 13-, 14-, 15-, or 16-membered fused tricycle is not

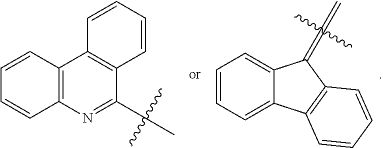 [SMILES: *c1nc2ccccc2c2ccccc12 ; *=C1c2ccccc2-c2ccccc21]

R_{S}′ preferably is (i) C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle; or (ii) 3- to 6-membered carbocycle or heterocycle, and wherein each 3- to 6-membered carbocycle or heterocycle in R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

In another embodiment, W is a 12-, 13-, 14-, 15-, or 16-membered fused tricycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —O—; Z₁ is N, Z₂ is C(H), Z₃ is CH₂, Z₄ is CH₂, and Z₅ is CH; L is —CH₂—CH₂—CH₂—CH₂—CH₂—; Y is —CH(N(R_{N})C(O)R_{S}′)— or —CH(N(R_{N})C(O)OR_{S}′)—; and [symbol] is —C(R₁R₂)—C(R₃R₄)—, wherein R₁, R₂, R₃ and R₄ are each independently hydrogen or halogen. Said 12-, 13-, 14-, 15-, or 16-membered fused tricycle can be either carbocycle or heterocycle. Preferably, said 12-, 13-, 14-, 15-, or 16-membered fused tricycle is not

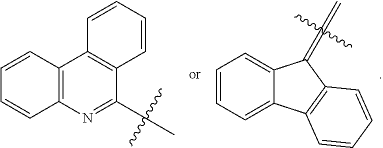 [SMILES: *c1nc2ccccc2c2ccccc12 ; *=C1c2ccccc2-c2ccccc21]

R_{S}′ preferably is (i) C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle; or (ii) 3- to 6-membered carbocycle or heterocycle, and wherein each 3- to 6-membered carbocycle or heterocycle in R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl. Preferably, R₁ and R₃ are hydrogen, and R₂ and R₄ are halogen (e.g., F). Also preferably, R₁, R₂, R₃ and R₄ are halogen (e.g., F).

In another embodiment, W is a 12-, 13-, 14-, 15-, or 16-membered fused tricycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —O—; Z₁ is N, Z₂ is C(H), Z₃ is CH₂, Z₄ is CH₂, and Z₅ is CH; L is —CH₂—CH₂—CH₂—CH₂—CH₂—; Y is —CH(N(R_{N})C(O)R_{S}′)—; and [symbol] is —CH═CH— or —C(R₁R₂)—C(R₃R₄)—, wherein R₁, R₂, R₃ and R₄ are each independently hydrogen or halogen. R_{S}′ is (i) hydrogen; (ii) C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle; or (iii) 3- to 6-membered carbocycle or heterocycle, and wherein each 3- to 6-membered carbocycle or heterocycle in R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl. Preferably, R_{S}′ is 5- to 6-membered carbocycle or heterocycle which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl. Said 12-, 13-, 14-, 15-, or 16-membered fused tricycle can be either carbocycle or heterocycle. Preferably, said 12-, 13-, 14-, 15-, or 16-membered fused tricycle is not

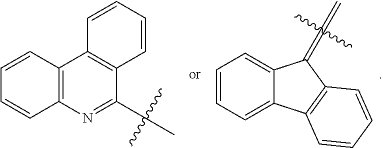 [SMILES: *c1nc2ccccc2c2ccccc12 ; *=C1c2ccccc2-c2ccccc21]

In one embodiment, W is a 9-, 10- or 11-membered fused bicycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —OC(O)— or —C(O)O—, and preferably, X is —OC(O)— wherein —O— is directly linked to Z₅, and —C(O)— is directly linked to W; Z₁ is N, Z₂ is C(R_{C}), Z₃ is C(R_{C})₂, Z₄ is C(R_{C})₂, and Z₅ is C(R_{C}); L is a straight C₄-C₆alkylene which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; Y is —CH(N(R_{N})C(O)R_{S}′)— or —CH(N(R_{N})C(O)OR_{S}′)—; and [symbol] is —CH═CH— or —C(R₁R₂)—C(R₃R₄)—, wherein R₁, R₂, R₃ and R₄ are each independently hydrogen or halogen. Said 9-, 10- or 11-membered fused bicycle can be either carbocycle or heterocycle. Preferably, said 9-, 10- or 11-membered fused bicycle is selected from quinoline, isoquinoline, quinoxaline or isoindoline.

In another embodiment, W is a 9-, 10- or 11-membered fused bicycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —OC(O)— or —C(O)O—, and preferably, X is —OC(O)— wherein —O— is directly linked to Z₅, and —C(O)— is directly linked to W; Z₁ is N, Z₂ is C(H), Z₃ is CH₂, Z₄ is CH₂, and Z₅ is CH; L is a straight C₄-C₆alkylene which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; Y is —CH(N(R_{N})_{C}(O)R_{S}′)— or —CH(N(R_{N})C(O)OR_{S}′)—; and [symbol] is —CH═CH— or —C(R₁R₂)—C(R₃R₄)—, wherein R₁, R₂, R₃ and R₄ are each independently hydrogen or halogen. Said 9-, 10- or 11-membered fused bicycle can be either carbocycle or heterocycle. Preferably, said 9-, 10- or 11-membered fused bicycle is selected from quinoline, isoquinoline, quinoxaline or isoindoline.

In another embodiment, W is a 9-, 10- or 11-membered fused bicycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —OC(O)— or —C(O)O—, and preferably, X is —OC(O)— wherein —O— is directly linked to Z₅, and —C(O)— is directly linked to W; Z₁ is N, Z₂ is C(H), Z₃ is CH₂, Z₄ is CH₂, and Z₅ is CH; L is a straight C₅alkylene, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; Y is —CH(N(R_{N})C(O)R_{S}′)— or —CH(N(R_{N})C(O)OR_{S}′)—; and [symbol] is —CH═CH— or —C(R₁R₂)—C(R₃R₄)—, wherein R₁, R₂, R₃ and R₄ are each independently hydrogen or halogen. Said 9-, 10- or 11-membered fused bicycle can be either carbocycle or heterocycle. Preferably, said 9-, 10- or 11-membered fused bicycle is selected from quinoline, isoquinoline, quinoxaline or isoindoline.

In another embodiment, W is a 9-, 10- or 11-membered fused bicycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —OC(O)— or —C(O)O—, and preferably, X is —OC(O)— wherein —O— is directly linked to Z₅, and —C(O)— is directly linked to W; Z₁ is N, Z₂ is C(H), Z₃ is CH₂, Z₄ is CH₂, and Z₅ is CH; L is —CH₂—CH₂—CH₂—CH₂—CH₂—; Y is —CH(N(R_{N})C(O)R_{S}′)— or —CH(N(R_{N})C(O)OR_{S}′)—; and [symbol] is —CH═CH— or —C(R₁R₂)—C(R₃R₄)—, wherein R₁, R₂, R₃ and R₄ are each independently hydrogen or halogen. Said 9-, 10- or 11-membered fused bicycle can be either carbocycle or heterocycle. Preferably, said 9-, 10- or 11-membered fused bicycle is selected from quinoline, isoquinoline, quinoxaline or isoindoline.

In another embodiment, W is a 9-, 10- or 11-membered fused bicycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —OC(O)— or —C(O)O—, and preferably, X is —OC(O)— wherein —O— is directly linked to Z₅, and —C(O)— is directly linked to W; Z₁ is N, Z₂ is C(H), Z₃ is CH₂, Z₄ is CH₂, and Z₅ is CH; L is —CH₂—CH₂—CH₂—CH₂—CH₂—; Y is —CH(N(R_{N})C(O)R_{S}′)— or —CH(N(R_{N})C(O)OR_{S}′)—; and [symbol] is —CH═CH—. Said 9-, 10- or 11-membered fused bicycle can be either carbocycle or heterocycle. Preferably, said 9-, 10- or 11-membered fused bicycle is selected from quinoline, isoquinoline, quinoxaline or isoindoline. R_{S}′ preferably is (i) C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle; or (ii) 3- to 6-membered carbocycle or heterocycle, and wherein each 3- to 6-membered carbocycle or heterocycle in R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

In another embodiment, W is a 9-, 10- or 11-membered fused bicycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —OC(O)— or —C(O)O—, and preferably, X is —OC(O)— wherein —O— is directly linked to Z₅, and —C(O)— is directly linked to W; Z₁ is N, Z₂ is C(H), Z₃ is CH₂, Z₄ is CH₂, and Z₅ is CH; L is —CH₂—CH₂—CH₂—CH₂—CH₂—; Y is —CH(N(R_{N})C(O)R_{S}′)— or —CH(N(R_{N})C(O)OR_{S}′)—; and [symbol] is —C(R₁R₂)—C(R₃R₄)—, wherein R₁, R₂, R₃ and R₄ are each independently hydrogen or halogen. Said 9-, 10- or 11-membered fused bicycle can be either carbocycle or heterocycle. Preferably, said 9-, 10- or 11-membered fused bicycle is selected from quinoline, isoquinoline, quinoxaline or isoindoline. R_{S}′ preferably is (i) C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle; or (ii) 3- to 6-membered carbocycle or heterocycle, and wherein each 3- to 6-membered carbocycle or heterocycle in R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl. Preferably, R₁ and R₃ are hydrogen, and R₂ and R₄ are halogen (e.g., F). Also preferably, R₁, R₂, R₃ and R₄ are halogen (e.g., F).

In another embodiment, W is a 9-, 10- or 11-membered fused bicycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —OC(O)— or —C(O)O—, and preferably, X is —OC(O)— wherein —O— is directly linked to Z₅, and —C(O)— is directly linked to W; Z₁ is N, Z₂ is C(H), Z₃ is CH₂, Z₄ is CH₂, and Z₅ is CH; L is —CH₂—CH₂—CH₂—CH₂—CH₂—; Y is —CH(N(R_{N})C(O)R_{S}′)—; and [symbol] is —CH═CH— or —C(R₁R₂)—C(R₃R₄)—, wherein R₁, R₂, R₃ and R₄ are each independently hydrogen or halogen. R_{S}′ is (i) hydrogen; (ii) C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle; or (iii) 3- to 6-membered carbocycle or heterocycle, and wherein each 3- to 6-membered carbocycle or heterocycle in R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl. Preferably, R_{S}′ is C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle. Said 9-, 10- or 11-membered fused bicycle can be either carbocycle or heterocycle. Preferably, said 9-, 10- or 11-membered fused bicycle is selected from quinoline, isoquinoline, quinoxaline or isoindoline.

In another embodiment, W is a 12-, 13-, 14-, 15-, or 16-membered fused tricycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —OC(O)— or —C(O)O—, and preferably, X is —OC(O)— wherein —O— is directly linked to Z₅, and —C(O)— is directly linked to W; Z₁ is N, Z₂ is C(R_{C}), Z₃ is C(R_{C})₂, Z₄ is C(R_{C})₂, and Z₅ is C(R_{C}); L is a straight C₄-C₆alkylene which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; Y is —CH(N(R_{N})C(O)R_{S}′)— or —CH(N(R_{N})C(O)OR_{S}′)—; and [symbol] is —CH═CH— or —C(R₁R₂)—C(R₃R₄)—, wherein R₁, R₂, R₃ and R₄ are each independently hydrogen or halogen. Said 12-, 13-, 14-, 15-, or 16-membered fused tricycle can be either carbocycle or heterocycle. Preferably, said 12-, 13-, 14-, 15-, or 16-membered fused tricycle is not

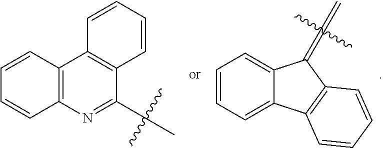 [SMILES: *c1nc2ccccc2c2ccccc12 ; *=C1c2ccccc2-c2ccccc21]

In another embodiment, W is a 12-, 13-, 14-, 15-, or 16-membered fused tricycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —OC(O)— or —C(O)O—, and preferably, X is —OC(O)— wherein —O— is directly linked to Z₅, and —C(O)— is directly linked to W; Z₁ is N, Z₂ is C(H), Z₃ is CH₂, Z₄ is CH₂, and Z₅ is CH; L is a straight C₄-C₆alkylene which is optionally substituted with one or more substituents selected from halogen, hydroxy, mercapto, amino, carboxy, nitro, oxo, phosphonoxy, phosphono, thioxo, formyl, cyano, C₂-C₆alkenyl, C₂-C₆alkynyl, C₁-C₆haloalkyl, C₂-C₆haloalkenyl or C₂-C₆haloalkynyl; Y is —CH(N(R_{N})C(O)R_{S}′)— or —CH(N(R_{N})C(O)OR_{S}′)—; and [symbol] is —CH═CH— or —C(R₁R₂)—C(R₃R₄)—, wherein R₁, R₂, R₃ and R₄ are each independently hydrogen or halogen. Said 12-, 13-, 14-, 15-, or 16-membered fused tricycle can be either carbocycle or heterocycle. Preferably, said 12-, 13-, 14-, 15-, or 16-membered fused tricycle is not

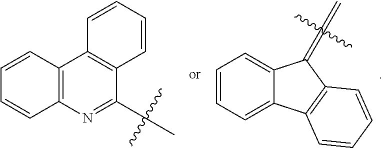 [SMILES: *c1nc2ccccc2c2ccccc12 ; *=C1c2ccccc2-c2ccccc21]

In another embodiment, W is a 12-, 13-, 14-, 15-, or 16-membered fused tricycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —OC(O)— or —C(O)O—, and preferably, X is —OC(O)— wherein —O— is directly linked to Z₅, and —C(O)— is directly linked to W; Z₁ is N, Z₂ is C(H), Z₃ is CH₂, Z₄ is CH₂, and Z₅ is CH; L is a straight C₅alkylene, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; Y is —CH(N(R_{N})C(O)R_{S}′)— or —CH(N(R_{N})C(O)OR_{S}′)—; and [symbol] is —CH═CH— or —C(R₁R₂)—C(R₃R₄)—, wherein R₁, R₂, R₃ and R₄ are each independently hydrogen or halogen. Said 12-, 13-, 14-, 15-, or 16-membered fused tricycle can be either carbocycle or heterocycle. Preferably, said 12-, 13-, 14-, 15-, or 16-membered fused tricycle is not

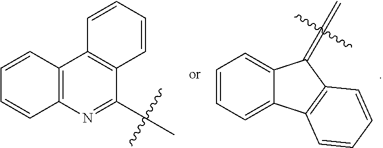 [SMILES: *c1nc2ccccc2c2ccccc12 ; *=C1c2ccccc2-c2ccccc21]

In another embodiment, W is a 12-, 13-, 14-, 15-, or 16-membered fused tricycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —OC(O)— or —C(O)O—, and preferably, X is —OC(O)— wherein —O— is directly linked to Z₅, and —C(O)— is directly linked to W; Z₁ is N, Z₂ is C(H), Z₃ is CH₂, Z₄ is CH₂, and Z₅ is CH; L is —CH₂—CH₂—CH₂—CH₂—CH₂—; Y is —CH(N(R_{N})C(O)R_{S}′)— or —CH(N(R_{N})C(O)OR_{S}′)—; and [symbol] is —CH═CH— or —C(R₁R₂)—C(R₃R₄)—, wherein R₁, R₂, R₃ and R₄ are each independently hydrogen or halogen. Said 12-, 13-, 14-, 15-, or 16-membered fused tricycle can be either carbocycle or heterocycle. Preferably, said 12-, 13-, 14-, 15-, or 16-membered fused tricycle is not

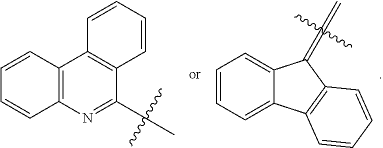 [SMILES: *c1nc2ccccc2c2ccccc12 ; *=C1c2ccccc2-c2ccccc21]

In another embodiment, W is a 12-, 13-, 14-, 15-, or 16-membered fused tricycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —OC(O)— or —C(O)O—, and preferably, X is —OC(O)— wherein —O— is directly linked to Z₅, and —C(O)— is directly linked to W; Z₁ is N, Z₂ is C(H), Z₃ is CH₂, Z₄ is CH₂, and Z₅ is CH; L is —CH₂—CH₂—CH₂—CH₂—CH₂—; Y is —CH(N(R_{N})C(O)R_{S}′)— or —CH(N(R_{N})C(O)OR_{S}′)—; and [symbol] is —CH═CH—. Said 12-, 13-, 14-, 15-, or 16-membered fused tricycle can be either carbocycle or heterocycle. Preferably, said 12-, 13-, 14-, 15-, or 16-membered fused tricycle is not

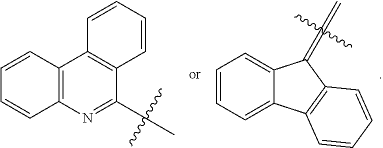 [SMILES: *c1nc2ccccc2c2ccccc12 ; *=C1c2ccccc2-c2ccccc21]

R_{S}′ preferably is (i) C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle; or (ii) 3- to 6-membered carbocycle or heterocycle, and wherein each 3- to 6-membered carbocycle or heterocycle in R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

In another embodiment, W is a 12-, 13-, 14-, 15-, or 16-membered fused tricycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —OC(O)— or —C(O)O—, and preferably, X is —OC(O)— wherein —O— is directly linked to Z₅, and —C(O)— is directly linked to W; Z₁ is N, Z₂ is C(H), Z₃ is CH₂, Z₄ is CH₂, and Z₅ is CH; L is —CH₂—CH₂—CH₂—CH₂—CH₂—; Y is —CH(N(R_{N})C(O)R_{S}′)— or —CH(N(R_{N})C(O)OR_{S}′)—; and [symbol] is —C(R₁R₂)—C(R₃R₄)—, wherein R₁, R₂, R₃ and R₄ are each independently hydrogen or halogen. Said 12-, 13-, 14-, 15-, or 16-membered fused tricycle can be either carbocycle or heterocycle. Preferably, said 12-, 13-, 14-, 15-, or 16-membered fused tricycle is not

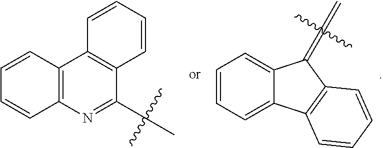 [SMILES: *c1nc2ccccc2c2ccccc12 ; *=C1c2ccccc2-c2ccccc21]

R_{S}′ preferably is (i) C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle; or (ii) 3- to 6-membered carbocycle or heterocycle, and wherein each 3- to 6-membered carbocycle or heterocycle in R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl. Preferably, R₁ and R₃ are hydrogen, and R₂ and R₄ are halogen (e.g., F). Also preferably, R₁, R₂, R₃ and R₄ are halogen (e.g., F).

In another embodiment, W is a 12-, 13-, 14-, 15-, or 16-membered fused tricycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; X is —OC(O)— or —C(O)O—, and preferably, X is —OC(O)— wherein —O— is directly linked to Z₅, and —C(O)— is directly linked to W; Z₁ is N, Z₂ is C(H), Z₃ is CH₂, Z₄ is CH₂, and Z₅ is CH; L is —CH₂—CH₂—CH₂—CH₂—CH₂—; Y is —CH(N(R_{N})C(O)OR_{S}′)—; and [symbol] is —CH═CH— or —C(R₁R₂)—C(R₃R₄)—, wherein R₁, R₂, R₃ and R₄ are each independently hydrogen or halogen. R_{S}′ is (i) hydrogen; (ii) C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle; or (iii) 3- to 6-membered carbocycle or heterocycle, and wherein each 3- to 6-membered carbocycle or heterocycle in R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl. Preferably, R_{S}′ is C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle. Said 12-, 13-, 14-, 15-, or 16-membered fused tricycle can be either carbocycle or heterocycle. Preferably, said 12-, 13-, 14-, 15-, or 16-membered fused tricycle is not

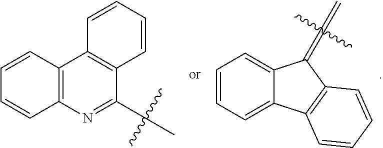 [SMILES: *c1nc2ccccc2c2ccccc12 ; *=C1c2ccccc2-c2ccccc21]

In yet another embodiment, a compound of Formula I is

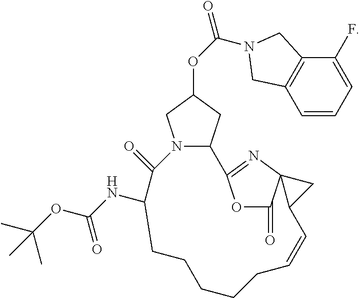 [SMILES: CC(C)(C)OC(=O)NC1CCCCC/C=C\C2CC23N=C(OC3=O)C2CC(OC(=O)N3Cc4cccc(F)c4C3)CN2C1=O]

The invention also features salts thereof

In still another embodiment, a compound of Formula I is

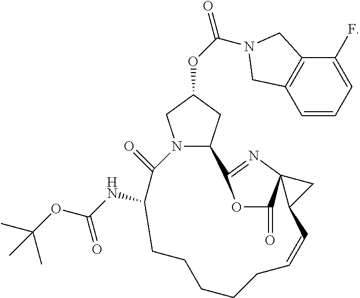 [SMILES: CC(C)(C)OC(=O)N[C@H]1CCCCC/C=C\[C@@H]2C[C@@]23N=C(OC3=O)[C@@H]2C[C@@H](OC(=O)N3Cc4cccc(F)c4C3)CN2C1=O]

The invention also features salts thereof.

In still another embodiment, a compound of Formula I is

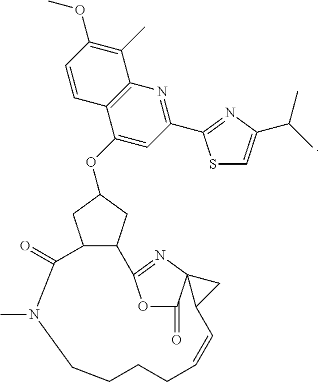 [SMILES: COc1ccc2c(OC3CC4C(=O)N(C)CCCC/C=C\C5CC56N=C(OC6=O)C4C3)cc(-c3nc(C(C)C)cs3)nc2c1C]

The invention also features salts thereof.

In still yet another embodiment, a compound of Formula I is

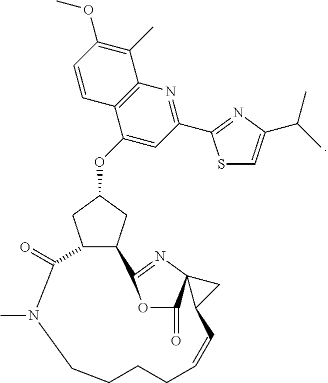 [SMILES: COc1ccc2c(O[C@H]3C[C@H]4C(=O)N(C)CCCC/C=C\[C@@H]5C[C@@]56N=C(OC6=O)[C@@H]4C3)cc(-c3nc(C(C)C)cs3)nc2c1C]

The invention also features salts thereof.

In still another embodiment, a compound of Formula I is

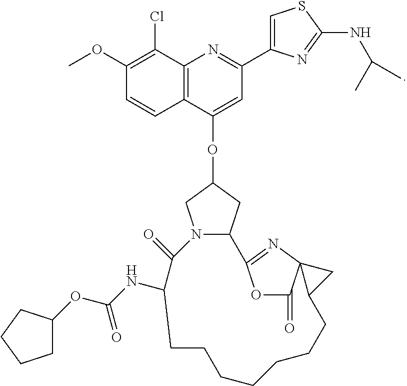 [SMILES: COc1ccc2c(OC3CC4C5=NC6(CC6CCCCCCCC(NC(=O)OC6CCCC6)C(=O)N4C3)C(=O)O5)cc(-c3csc(NC(C)C)n3)nc2c1Cl]

The invention also features salts thereof.

In still yet another embodiment, a compound of Formula I is

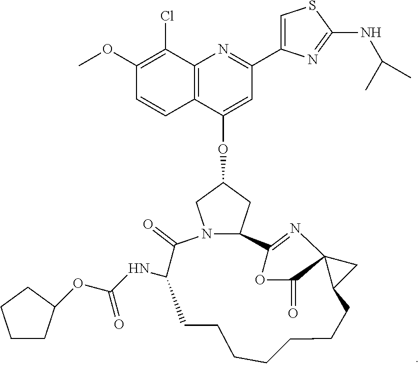 [SMILES: COc1ccc2c(O[C@@H]3C[C@H]4C5=N[C@@]6(C[C@H]6CCCCCCC[C@H](NC(=O)OC6CCCC6)C(=O)N4C3)C(=O)O5)cc(-c3csc(NC(C)C)n3)nc2c1Cl]

The invention also features salts thereof.

In still another embodiment, a compound of Formula I is

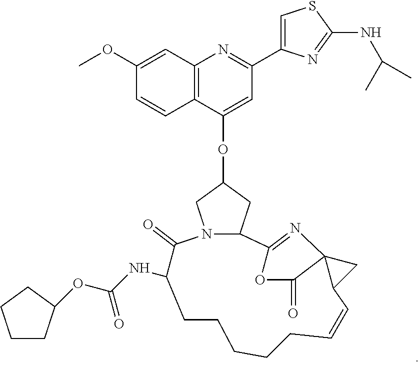 [SMILES: COc1ccc2c(OC3CC4C5=NC6(CC6/C=C\CCCCCC(NC(=O)OC6CCCC6)C(=O)N4C3)C(=O)O5)cc(-c3csc(NC(C)C)n3)nc2c1]

The invention also features salts thereof

In still yet another embodiment, a compound of Formula I is

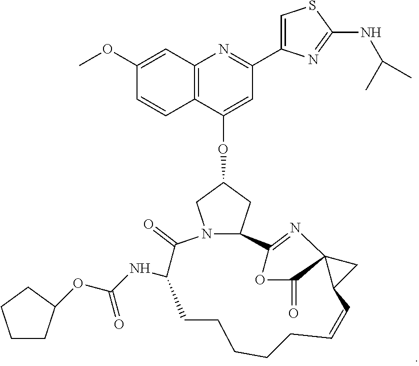 [SMILES: COc1ccc2c(O[C@@H]3C[C@H]4C5=N[C@@]6(C[C@H]6/C=C\CCCCC[C@H](NC(=O)OC6CCCC6)C(=O)N4C3)C(=O)O5)cc(-c3csc(NC(C)C)n3)nc2c1]

The invention also features salts thereof.

In any aspect, embodiment, example, preference and description of the invention, Formula I preferably has the stereochemistry depicted in Formula I′, wherein Y in Formula I is C(R_{A}R_{B}).

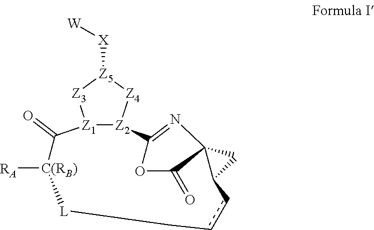

In any aspect, embodiment, example, preference and description of the invention, Y and W in Formula I, or R_{A} and W in Formula I′, can be covalently linked through a linker LL, as depicted in Formula II and Formula II′, respectively. LL is an optionally substituted C₃-C₁₂alkylene, C₃-C₁₂alkenylene or C₃-C₁₂alkynylene, each of said C₃-C₁₂alkylene, C₃-C₁₂alkenylene or C₃-C₁₂alkynylene optionally containing 1, 2, or 3 heteroatoms independently selected from O, S or N, and two adjacent substituents on LL can optionally form an optionally substituted carbocycle or heterocycle.

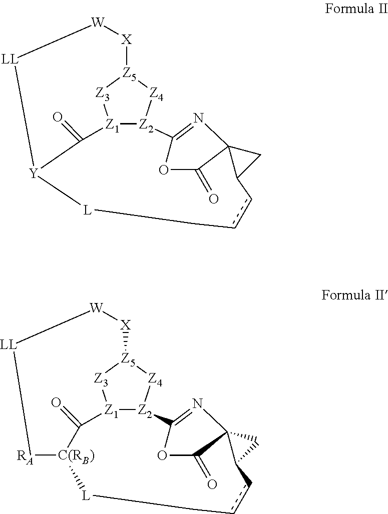

Compounds of Formula I can be prepared according to Scheme I. A compound of Formula Ia is treated with an acid activation agent (e.g., N,N-carbonyldiimidazole (CDI)) to form a compound of Formula Ib, wherein W, X, Z₁, Z₂, Z₃, Z₄, Z₅, Y, L, and [symbol] are as defined above, and V is a leaving group (e.g.

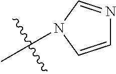 [SMILES: *n1ccnc1]

when CDI is used). The compound of Formula Ib is then treated with a base to form a compound of Formula I. Suitable bases for this purpose include, but are not limited to, organic bases (e.g., diazabicycloundene (DBU), tetramethylguanidine (TMG), dimethylaminopyridine (DMAP), or like reagents), inorganic bases (e.g., metal carbonates, metal phosphates, or like reagents), metal amide bases (e.g., metal diisopropylamide (MDA), metal hexamethyldisilylamide (MHMDS), or like reagents), metal alkoxide bases (e.g., metal t-butoxides or like reagents), organo metal bases (e.g., n-butyl lithium, isopropyl magnesium chloride, or like reagents), metal hydride bases (e.g., sodium hydride or like reagents), with DBU being preferred.

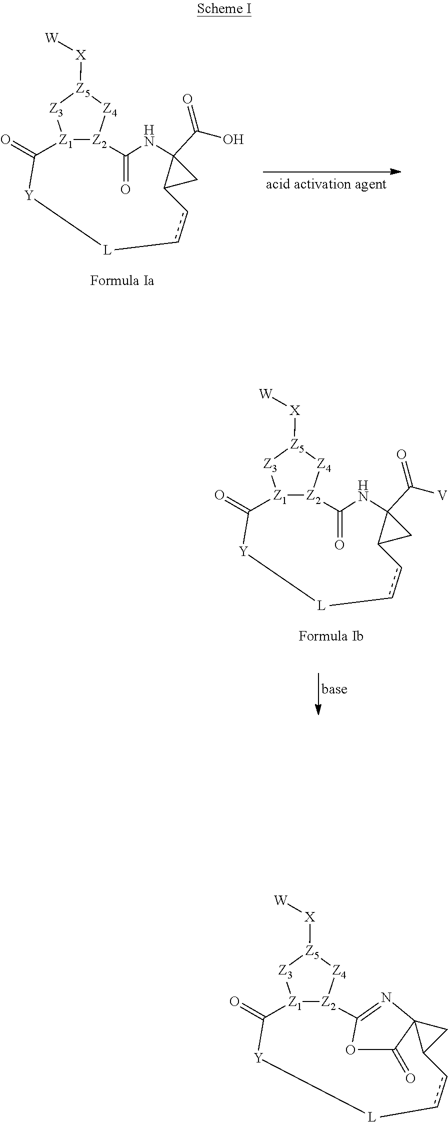

Derivatization of the carboxylic acid OH group in Formula Ia into the leaving group V in Formula Ib can also be achieved using other acid activation agents. For example, a compound of Formula Ia can be treated with SOV₂, (COV)₂, COV₂, POV₃, or like reagents to produce a compound of Formula Ib wherein V is an acid halide (e.g., V=F, Cl, Br, I); for another example, a compound of Formula Ia can be treated with an acid halide (e.g., pivaloyl chloride, acetyl chloride, etc.), or a carboxylic acid anhydride (e.g., pivalic anhydride, acetic anhydride, etc.), or a haloformate (e.g., isobutyl chloroformate, etc.), or like reagents to produce a compound of Formula Ib wherein V is a mixed anhydride (e.g., V=—OC(O)R); for yet another example, a compound of Formula Ia can be treated with n-propyl phosphonic acid anhydride, diphenylphosphoryl azide or like reagents to produce a compound of Formula Ib wherein V is an acyl phosphate (e.g., V=—OP(O)Y₂); for still yet another example, a compound of Formula Ib can be a thiol ester (e.g., V=S—R); for yet another example, a compound of Formula Ia can be treated with dicyclohexylcarbodiimide or like reagents in combination with hydroxysuccinimide, hydroxybenzotriazole or like reagents to produce a compound of Formula Ib wherein V is an activated ester (V=—OR); for yet another example, a compound of Formula Ia can be treated with 2-chloro-4,6-dimethoxy-1,3,5-triazine, cyanuric chloride, or like reagents to produce a compound of Formula Ib wherein V is an aryl ester (V=—O-aryl). These compounds of Formula Ib can then be treated with a suitable base as described above to form a compound of Formula I.

Compounds of Formulas I′, II and II′ can be similarly prepared according to Schemes I′. II and II′, respectively, wherein the acid activation agent and the base are as described in Scheme I, and all other variables are as described above.

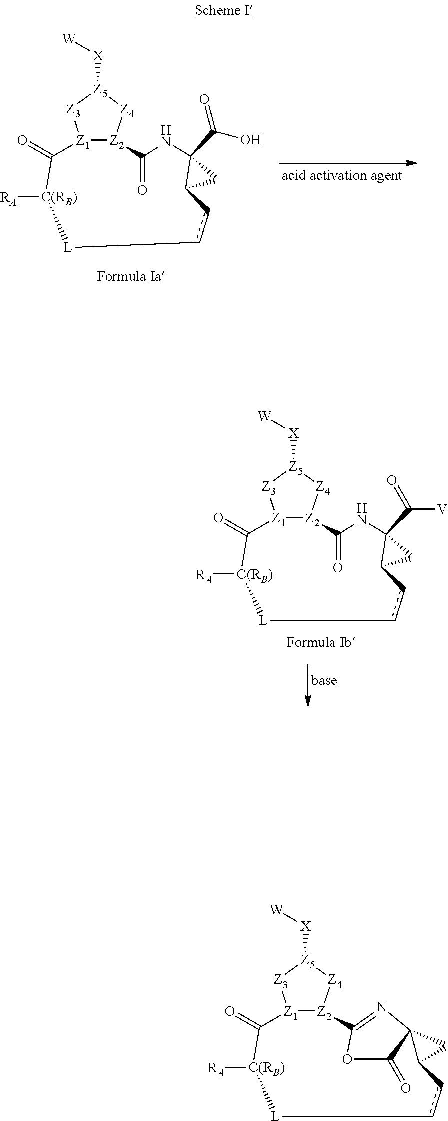

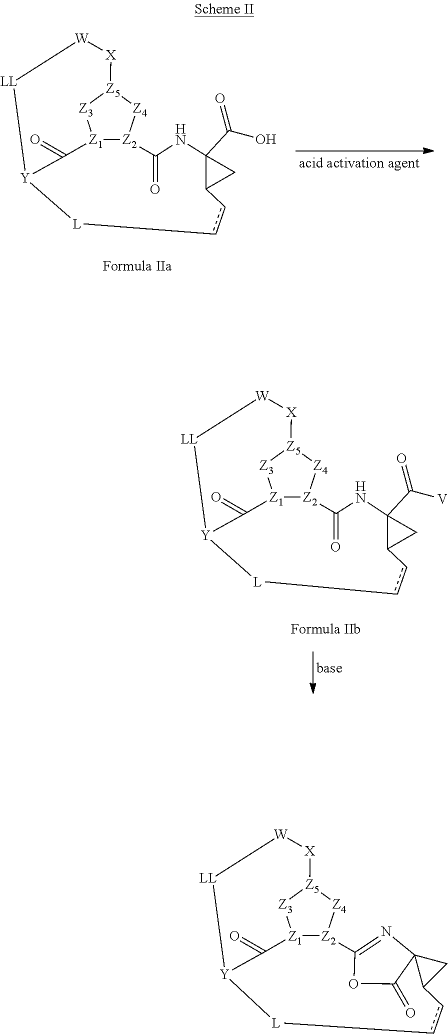

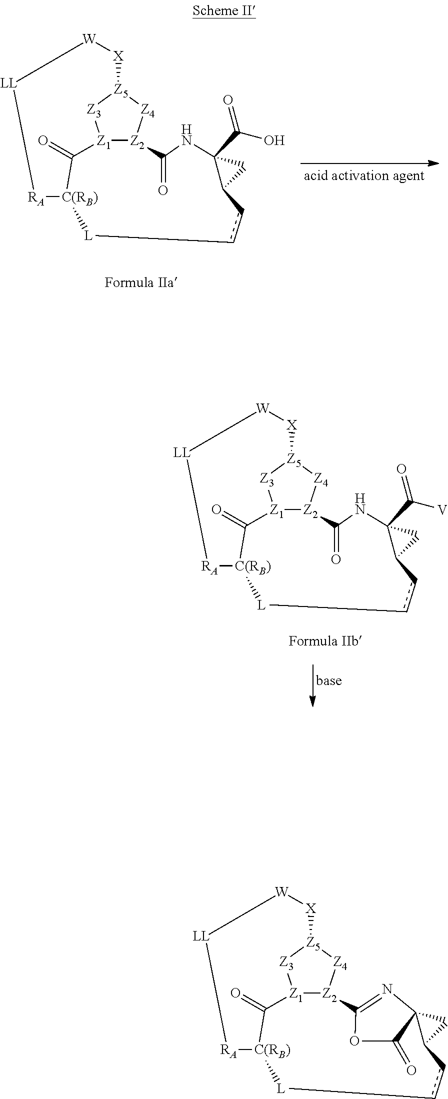

In another aspect, the present invention features a reaction solution comprising a compound of Formula I, I′, II or II′ as described above. Said compound can be any compound of Formula I, I′, II or II′ described or contemplated under any embodiment, example or preference described above. In one embodiment, the reaction solution comprises at least 1% by weight of said compound. In another embodiment, the reaction solution comprises at least 2% by weight of said compound. In still another embodiment, the reaction solution comprises at least 3% by weight of said compound. In still another embodiment, the reaction solution comprises at least 4% by weight of said compound. In still another embodiment, the reaction solution comprises at least 5% by weight of said compound. In still another embodiment, the reaction solution comprises at least 6% by weight of said compound. In still another embodiment, the reaction solution comprises at least 7% by weight of said compound. In still another embodiment, the reaction solution comprises at least 8% by weight of said compound. In still another embodiment, the reaction solution comprises at least 9% by weight of said compound. In still another embodiment, the reaction solution comprises at least 10% by weight of said compound. In still another embodiment, the reaction solution comprises at least 15% by weight of said compound. In still another embodiment, the reaction solution comprises at least 20% by weight of said compound. In still another embodiment, the reaction solution comprises at least 25% by weight of said compound. In still another embodiment, the reaction solution comprises at least 30% by weight of said compound. In still another embodiment, the reaction solution comprises at least 35% by weight of said compound. In still another embodiment, the reaction solution comprises at least 40% by weight of said compound. In still another embodiment, the reaction solution comprises at least 45% by weight of said compound. In still another embodiment, the reaction solution comprises at least 50% by weight of said compound.

In still another embodiment, at least 1% by weight of all solutes in the reaction solution is said compound. In still another embodiment, at least 2% by weight of all solutes in the reaction solution is said compound. In still another embodiment, at least 3% by weight of all solutes in the reaction solution is said compound. In still another embodiment, at least 4% by weight of all solutes in the reaction solution is said compound. In still another embodiment, at least 5% by weight of all solutes in the reaction solution is said compound. In still another embodiment, at least 6% by weight of all solutes in the reaction solution is said compound. In still another embodiment, at least 7% by weight of all solutes in the reaction solution is said compound. In still another embodiment, at least 8% by weight of all solutes in the reaction solution is said compound. In still another embodiment, at least 9% by weight of all solutes in the reaction solution is said compound. In still another embodiment, at least 10% by weight of all solutes in the reaction solution is said compound. In still another embodiment, at least 15% by weight of all solutes in the reaction solution is said compound. In still another embodiment, at least 20% by weight of all solutes in the reaction solution is said compound. In still another embodiment, at least 25% by weight of all solutes in the reaction solution is said compound. In still another embodiment, at least 30% by weight of all solutes in the reaction solution is said compound. In still another embodiment, at least 35% by weight of all solutes in the reaction solution is said compound. In still another embodiment, at least 40% by weight of all solutes in the reaction solution is said compound. In still another embodiment, at least 45% by weight of all solutes in the reaction solution is said compound. In still another embodiment, at least 50% by weight of all solutes in the reaction solution is said compound.

In yet another aspect, the present invention features methods of making HCV protease inhibitors. The methods comprise reacting a compound of Formula I with NH(R_{M})-G to form a compound of Formula A,

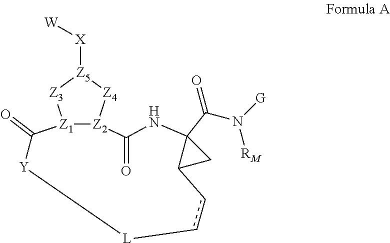

wherein G is —R_{T}, —C(O)R_{T}, —SO₂R_{T}, —S(O)R_{T}, —SO₂N(R_{N})R_{T}, —S(O)N(R_{N})R_{T} or —C(O)OR_{T}, and R_{M} is R_{N}, and R_{T} is R_{S}, and wherein W, X, Z₁, Z₂, Z₃, Z₄, Z₅, Y, L, R_{N}, R_{S}, and [symbol] are as defined in Formula I. Any compound of Formula I described or contemplated under any embodiment, example and preference described above can be reacted with NH(R_{M})-G to make a corresponding compound of Formula A.

Preferably, R_{M} is hydrogen, G is —SO₂R_{T}, and R_{T} is cyclopropyl which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl. More preferably, R_{M} is hydrogen, G is —SO₂R_{T}, and R_{T} is cyclopropyl.

Preferably, a compound of Formula I is reacted with NH(R_{M})-G in the presence of a base, such as a strong organic base (e.g., diazabicycloundene (DBU), tetramethylguanidine (TMG), or like reagents), an inorganic base (e.g., metal carbonates, metal phosphates, or like reagents), a metal amide base (e.g., metal diisopropylamide (MDA), metal hexamethyldisilylamide (MHMDS), or like reagents), or a metal alkoxide bases (e.g., metal t-butoxides or like reagents), with DBU being preferred.

In one embodiment, the compound of Formula A is

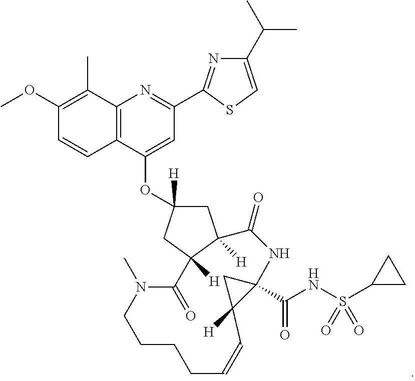 [SMILES: COc1ccc2c(O[C@@H]3C[C@H]4C(=O)N[C@]5(C(=O)NS(=O)(=O)C6CC6)C[C@H]5/C=C\CCCCN(C)C(=O)[C@@H]4C3)cc(-c3nc(C(C)C)cs3)nc2c1C]

In yet another aspect, the present invention features methods of making HCV protease inhibitors. The methods comprise reacting a compound of Formula I′ with NH(R_{M})-G to form a compound of Formula A′,

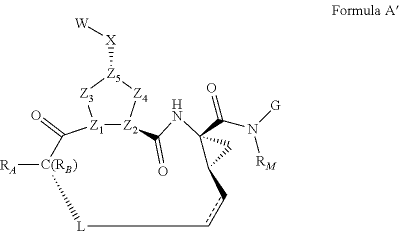

wherein G is —R_{T}, —C(O)R_{T}, —SO₂R_{T}, —S(O)R_{T}, —SO₂N(R_{N})R_{T}, —S(O)N(R_{N})R_{T} or —C(O)OR_{T}, and R_{M} is R_{N}, and R_{T} is R_{S}, and wherein W, X, Z₁, Z₂, Z₃, Z₄, Z₅, Y, L, R_{N}, R_{S}, R_{A}, R_{B}, and [symbol] are as defined in Formula I′. Any compound of Formula I′ described or contemplated under any embodiment, example and preference described above can be reacted with NH(R_{M})-G to make a corresponding compound of Formula A′.

Preferably, R_{M} is hydrogen, G is —SO₂R_{T}, and R_{T} is cyclopropyl which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl. More preferably, R_{M} is hydrogen, G is —SO₂R_{T}, and R_{T} is cyclopropyl.

Preferably, a compound of Formula I′ is reacted with NH(R_{M})-G in the presence of a base, such as a strong organic base (e.g., diazabicycloundene (DBU), tetramethylguanidine (TMG), or like reagents), an inorganic base (e.g., metal carbonates, metal phosphates, or like reagents), a metal amide base (e.g., metal diisopropylamide (MDA), metal hexamethyldisilylamide (MHMDS), or like reagents), or a metal alkoxide bases (e.g., metal t-butoxides or like reagents), with DBU being preferred.

In one embodiment, the compound of Formula A′ is

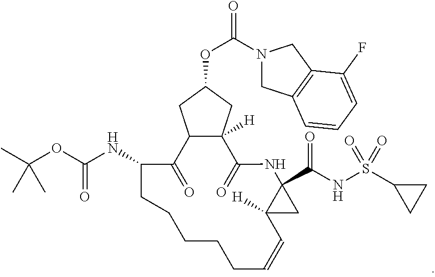 [SMILES: CC(C)(C)OC(=O)N[C@H]1CCCCC/C=C\[C@@H]2C[C@@]2(C(=O)NS(=O)(=O)C2CC2)NC(=O)[C@@H]2C[C@@H](OC(=O)N3Cc4cccc(F)c4C3)CC2C1=O]

In yet another aspect, the present invention features methods of making HCV protease inhibitors. The methods comprise reacting a compound of Formula II with NH(R_{M})-G to form a compound of Formula B,

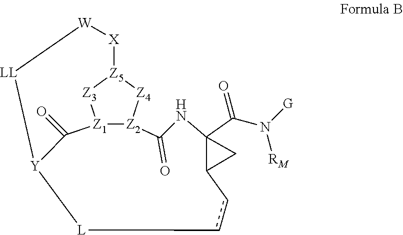

wherein G is —R_{T}, —C(O)R_{T}, —SO₂R_{T}, —S(O)R_{T}, —SO₂N(R_{N})R_{T}, —S(O)N(R_{N})R_{T} or —C(O)OR_{T}, and R_{M} is R_{N}, and R_{T} is R_{S}, and wherein W, X, Z₁, Z₂, Z₃, Z₄, Z₅, Y, L, LL, R_{N}, R_{S}, and [symbol] are as defined in Formula II. Any compound of Formula II described or contemplated under any embodiment, example and preference described above can be reacted with NH(R_{M})-G to make a corresponding compound of Formula B.

Preferably, R_{M} is hydrogen, G is —SO₂R_{T}, and R_{T} is cyclopropyl which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl. More preferably, R_{M} is hydrogen, G is —SO₂R_{T}, and R_{T} is cyclopropyl.

Preferably, a compound of Formula II is reacted with NH(R_{M})-G in the presence of a base, such as a strong organic base (e.g., diazabicycloundene (DBU), tetramethylguanidine (TMG), or like reagents), an inorganic base (e.g., metal carbonates, metal phosphates, or like reagents), a metal amide base (e.g., metal diisopropylamide (MDA), metal hexamethyldisilylamide (MHMDS), or like reagents), or a metal alkoxide bases (e.g., metal t-butoxides or like reagents), with DBU being preferred.

In yet another aspect, the present invention features methods of making HCV protease inhibitors. The methods comprise reacting a compound of Formula II′ with NH(R_{M})-G to form a compound of Formula B′,

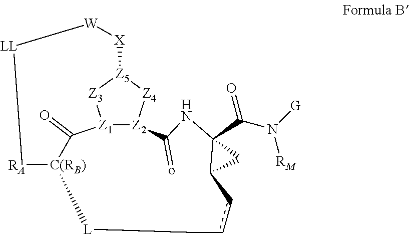

wherein G is —R_{T}, —C(O)R_{T}, —SO₂R_{T}, —S(O)R_{T}, —SO₂N(R_{N})R_{T}, —S(O)N(R_{N})R_{T} or —C(O)OR_{T}, and R_{M} is R_{N}, and R_{T} is R_{S}, and wherein W, X, Z₁, Z₂, Z₃, Z₄, Z₅, Y, L, LL, R_{N}, R_{S}, R_{A}, R_{B}, and [symbol] are as defined in Formula II′. Any compound of Formula II′ described or contemplated under any embodiment, example and preference described above can be reacted with NH(R_{M})-G to make a corresponding compound of Formula B′.

Preferably, R_{M} is hydrogen, G is —SO₂R_{T}, and R_{T} is cyclopropyl which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl. More preferably, R_{M} is hydrogen, G is —SO₂R_{T}, and R_{T} is cyclopropyl.

Preferably, a compound of Formula II′ is reacted with NH(R_{M})-G in the presence of a base, such as a strong organic base (e.g., diazabicycloundene (DBU), tetramethylguanidine (TMG), or like reagents), an inorganic base (e.g., metal carbonates, metal phosphates, or like reagents), a metal amide base (e.g., metal diisopropylamide (MDA), metal hexamethyldisilylamide (MHMDS), or like reagents), or a metal alkoxide bases (e.g., metal t-butoxides or like reagents), with DBU being preferred.

The compounds of Formulas I, I′, II and II′ can be used in the form of salts. Depending on the particular compound, a salt of a compound may be advantageous due to one or more of the salt's physical properties, such as enhanced stability under certain conditions or desired solubility in water or oil. In some instances, a salt of a compound may be useful for the isolation or purification of the compound.

Acid addition salts may be prepared from inorganic or organic acids. Examples of suitable inorganic acids include, but are not limited to, hydrochloric, hydrobromic, hydroionic, nitric, carbonic, sulfuric, and phosphoric acid. Examples of suitable organic acids include, but are not limited to, aliphatic, cycloaliphatic, aromatic, araliphatic, heterocyclyl, carboxylic, and sulfonic classes of organic acids. Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, b-hydroxybutyric acid, galactarate, galacturonate, adipate, alginate, bisulfate, butyrate, camphorate, camphorsulfonate, cyclopentanepropionate, dodecylsulfate, glycoheptanoate, glycerophosphate, hemisulfate, heptanoate, hexanoate, nicotinate, 2-naphthalesulfonate, oxalate, palmoate, pectinate, persulfate, 3-phenylpropionate, picrate, pivalate, thiocyanate, tosylate, and undecanoate.

Base addition salts include, but are not limited to, metallic salts and organic salts. Non-limiting examples of suitable metallic salts include alkali metal (group Ia) salts, alkaline earth metal (group Ba) salts, and other metal salts. Such salts may be made, without limitation, from aluminum, calcium, lithium, magnesium, potassium, sodium, or zinc. Non-limiting examples of suitable organic salts can be made from tertiary amines and quaternary amine, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups can be quaternized with agents such as alkyl halides (e.g., methyl, ethyl, propyl, butyl, decyl, lauryl, myristyl, and stearyl chlorides/bromides/iodides), dialkyl sulfates (e.g., dimethyl, diethyl, dibuytl, and diamyl sulfates), aralkyl halides (e.g., benzyl and phenethyl bromides), and others.

The compounds of Formulas I, I′, II and II′ may comprise asymmetrically substituted carbon atoms known as chiral centers. These compounds may exist, without limitation, as single stereoisomers (e.g., single enantiomers or single diastereomer), mixtures of stereoisomers (e.g. a mixture of enantiomers or diastereomers), or racemic mixtures. Compounds identified herein as single stereoisomers are meant to describe compounds that are present in a form that is substantially free from other stereoisomers (e.g., substantially free from other enantiomers or diastereomers). By “substantially free,” it means that at least 80% of the compound in a composition is the described stereoisomer; preferably, at least 90% of the compound in a composition is the described stereoisomer; and more preferably, at least 95%, 96%, 97%, 98% or 99% of the compound in a composition is the described stereoisomer. Where the stereochemistry of a chiral carbon is not specified in the chemical structure of a compound, the chemical structure is intended to encompass compounds containing either stereoisomer of the chiral center.

Individual stereoisomers of the compounds of this invention can be prepared using a variety of methods known in the art. These methods include, but are not limited to, stereospecific synthesis, chromatographic separation of diastereomers, chromatographic resolution of enantiomers, conversion of enantiomers in an enantiomeric mixture to diastereomers followed by chromatographically separation of the diastereomers and regeneration of the individual enantiomers, and enzymatic resolution.

Stereospecific synthesis typically involves the use of appropriate optically pure (enantiomerically pure) or substantial optically pure materials and synthetic reactions that do not cause racemization or inversion of stereochemistry at the chiral centers. Mixtures of stereoisomers of compounds, including racemic mixtures, resulting from a synthetic reaction may be separated, for example, by chromatographic techniques as appreciated by those of ordinary skill in the art. Chromatographic resolution of enantiomers can be accomplished by using chiral chromatography resins, many of which are commercially available. In a non-limiting example, racemate is placed in solution and loaded onto the column containing a chiral stationary phase. Enantiomers can then be separated by HPLC.

Resolution of enantiomers can also be accomplished by converting enantiomers in a mixture to diastereomers by reaction with chiral auxiliaries. The resulting diastereomers can be separated by column chromatography or crystallization/re-crystallization. This technique is useful when the compounds to be separated contain a carboxyl, amino or hydroxyl group that will form a salt or covalent bond with the chiral auxiliary. Non-limiting examples of suitable chiral auxiliaries include chirally pure amino acids, organic carboxylic acids or organosulfonic acids. Once the diastereomers are separated by chromatography, the individual enantiomers can be regenerated. Frequently, the chiral auxiliary can be recovered and used again.

Enzymes, such as esterases, phosphatases or lipases, can be useful for the resolution of derivatives of enantiomers in an enantiomeric mixture. For example, an ester derivative of a carboxyl group in the compounds to be separated can be treated with an enzyme which selectively hydrolyzes only one of the enantiomers in the mixture. The resulting enantiomerically pure acid can then be separated from the unhydrolyzed ester.

Alternatively, salts of enantiomers in a mixture can be prepared using any suitable method known in the art, including treatment of the carboxylic acid with a suitable optically pure base such as alkaloids or phenethylamine, followed by precipitation or crystallization/re-crystallization of the enantiomerically pure salts. Methods suitable for the resolution/separation of a mixture of stereoisomers, including racemic mixtures, can be found in ENANTIOMERS, RACEMATES, AND RESOLUTIONS (Jacques et al., 1981, John Wiley and Sons, New York, N.Y.).

A compound of this invention may possess one or more unsaturated carbon-carbon double bonds. All double bond isomers, such as the cis (Z) and trans (E) isomers, and mixtures thereof are intended to be encompassed within the scope of a recited compound unless otherwise specified. In addition, where a compound exists in various tautomeric forms, a recited compound is not limited to any one specific tautomer, but rather is intended to encompass all tautomeric forms.

Certain compounds of Formulas I, I′, II and II′ may exist in different stable conformational forms which may be separable. Torsional asymmetry due to restricted rotations about an asymmetric single bond, for example because of steric hindrance or ring strain, may permit separation of different conformers. The invention encompasses each conformational isomer of these compounds and mixtures thereof.

Certain compounds of Formulas I, I′, II and II′ may also exist in zwitterionic form and the invention encompasses each zwitterionic form of these compounds and mixtures thereof.

The compounds of Formulas I, I′, II and II′ are generally described herein using standard nomenclature. For a recited compound having asymmetric center(s), it should be understood that all of the stereoisomers of the compound and mixtures thereof are encompassed in the present invention unless otherwise specified. Non-limiting examples of stereoisomers include enantiomers, diastereomers, and cis-transisomers. Where a recited compound exists in various tautomeric forms, the compound is intended to encompass all tautomeric forms. Certain compounds are described herein using general formulas that include variables (e.g., W, X, Z₁, Z₂, Z₃, Z₄, Z₅, Y, L, R_{A}, R_{B}, R_{C}, R_{D}, R_{N} or R_{S}). Unless otherwise specified, each variable within such a formula is defined independently of any other variable, and any variable that occurs more than one time in a formula is defined independently at each occurrence. If moieties are described as being “independently” selected from a group, each moiety is selected independently from the other. Each moiety therefore can be identical to or different from the other moiety or moieties.

The number of carbon atoms in a hydrocarbyl moiety can be indicated by the prefix “Cₓ-C_{y},” where x is the minimum and y is the maximum number of carbon atoms in the moiety, or “C_{z}” wherein z is the number of carbon atoms in the moiety. Thus, for example, “C₁-C₆alkyl” refers to an alkyl substituent containing from 1 to 6 carbon atoms. Illustrating further, C₃-C₆cycloalkyl means a saturated hydrocarbyl ring containing from 3 to 6 carbon ring atoms. Unless otherwise specified, a prefix attached to a multiple-component substituent only applies to the first component that immediately follows the prefix. To illustrate, the term “carbocyclylalkyl” contains two components: carbocyclyl and alkyl. Thus, for example, C₃-C₆-carbocyclylC₁-C₆alkyl refers to a C₃-C₆-carbocyclyl appended to the parent molecular moiety through a C₁-C₆alkyl group.

Unless otherwise specified, when a linking element links two other elements in a depicted chemical structure, the leftmost-described component of the linking element is bound to the left element in the depicted structure, and the rightmost-described component of the linking element is bound to the right element in the depicted structure. To illustrate, if the chemical structure is W—X—Z₅ and X is —C(O)O—, then the chemical structure is —W—C(O)O—Z₅.

If a linking element in a depicted structure is absent, then the element left to the linking element is joined directly to the element right to the linking element via a covalent bond. For example, if a chemical structure is depicted as W—X—Z₅, and X is selected as absent, then the chemical structure will be W—Z₅. If two or more adjacent linking elements in a depicted structure are absent, then the element left to these linking elements is joined directly to the element right to these linking elements via a covalent bond.

When a chemical formula is used to describe a moiety, the dash(s) indicates the portion of the moiety that has the free valence(s).

If a moiety is described as being “optionally substituted”, the moiety is either substituted or unsubstituted. If a moiety is described as being optionally substituted with up to a particular number of non-hydrogen radicals, that moiety may be either unsubstituted, or substituted by up to that particular number of non-hydrogen radicals or by up to the maximum number of substitutable positions on the moiety, whichever is less. Thus, for example, if a moiety is described as a heterocycle optionally substituted with up to three non-hydrogen radicals, then any heterocycle with less than three substitutable positions will be optionally substituted by up to only as many non-hydrogen radicals as the heterocycle has substitutable positions. To illustrate, tetrazolyl (which has only one substitutable position) will be optionally substituted with up to one non-hydrogen radical. To illustrate further, if an amino nitrogen is described as being optionally substituted with up to two non-hydrogen radicals, then a primary amino nitrogen will be optionally substituted with up to two non-hydrogen radicals, whereas a secondary amino nitrogen will be optionally substituted with up to only one non-hydrogen radical.

Unless specified, the term “optionally substituted” refers to optional substitution by independent replacement of one, two, three or more of the hydrogen atoms with suitable substituents. Non-limiting examples of substituents include —F, —Cl, —Br, —I, hydroxy, protected hydroxy, —NO₂, —N₃, —CN, —NH₂, protected amino, oxo, thioxo, —NH—C₁-C₁₂-alkyl, —NH—C₂-C₈-alkenyl, —NH—C₂-C₈-alkynyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₈-alkenyl, —O—C₂-C₈-alkynyl, —O—C₃-C₁₂-cycloalkyl, —O-aryl, —O-heteroaryl, —O-heterocycloalkyl, —C(O)—C₁-C₁₂-alkyl, —C(O)—C₂-C₈-alkenyl, —C(O)—C₂-C₈-alkynyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₈-alkenyl, —CONH—C₂-C₈-alkynyl, —CONH—C₃-C₁₂-cycloalkyl, —CONH-aryl, —CONH-heteroaryl, —CONH-heterocycloalkyl, —OCO₂—C₁-C₁₂-alkyl, —OCO₂—C₂-C₈-alkenyl, —OCO₂—C₂-C₈-alkynyl, —OCO₂—C₃-C₁₂-cycloalkyl, —OCO₂-aryl, —OCO₂-heteroaryl, —OCO₂-heterocycloalkyl, —OCONH₂, —OCONH—C₁-C₁₂-alkyl, —OCONH—C₂-C₈-alkenyl, —OCONH—C₂-C₈-alkynyl, —OCONH—C₃-C₁₂-cycloalkyl, —OCONH-aryl, —OCONH-heteroaryl, —OCONH-heterocycloalkyl, —NHC(O)—C₁-C₁₂-alkyl, —NHC(O)—C₂-C₈-alkenyl, —NHC(O)—C₂-C₈-alkynyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₈-alkenyl, —NHCO₂—C₂-C₈-alkynynl, —NHCO₂—C₃-C₁₂-cycloalkyl, —NHCO₂-aryl, —NHCO₂-heteroaryl, —NHCO₂-heterocycloalkyl, —NHC(O)NH₂, —NHC(O)NH—C₁-C₁₂-alkyl, —NHC(O)NH—C₂-C₈-alkenyl, —NHC(O)NH—C₂-C₈-alkynyl, —NHC(O)NH—C₃-C₁₂-cycloalkyl, —NHC(O)NH-aryl, —NHC(O)NH-heteroaryl, —NHC(O)NH-heterocycloalkyl, NHC(S)NH₂, —NHC(S)NH—C₁-C₁₂-alkyl, —NHC(S)NH—C₂-C₈-alkenyl, —NHC(S)NH—C₂-C₈-alkynyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₈-alkenyl, —NHC(NH)NH—C₂-C₈-alkynyl, —NHC(NH)NH—C₃-C₁₂-cycloalkyl, —NHC(NH)NH-aryl, —NHC(NH)NH-heteroaryl, —NHC(NH)NH-heterocycloalkyl, —NHC(NH)—C₁-C₁₂-alkyl, —NHC(NH)—C₂-C₈-alkenyl, —NHC(NH)—C₂-C₈-alkynyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₈-alkenyl, —C(NH)NH—C₂-C₈-alkynyl, —C(NH)NH—C₃-C₁₂-cycloalkyl, —C(NH)NH-aryl, —C(NH)NH-heteroaryl, —C(NH)NH-heterocycloalkyl, —S(O)—C₁-C₁₂-alkyl, —S(O)—C₂-C₈-alkenyl, —S(O)—C₂-C₈-alkynyl, —S(O)—C₃-C₁₂-cycloalkyl, —S(O)-aryl, —S(O)-heteroaryl, —S(O)-heterocycloalkyl, —SO₂NH₂, —SO₂NH—C₁-C₁₂-alkyl, —SO₂NH—C₂-C₈-alkenyl, —SO₂NH—C₂-C₈-alkynyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₈-alkenyl, —NHSO₂—C₂-C₈-alkynyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₈-alkenyl, —S—C₂-C₈-alkynyl, —S—C₃-C₁₂-cycloalkyl, —S-aryl, -heteroaryl, —S-heterocycloalkyl, or methylthiomethyl.

Where a moiety is substituted with oxo or thioxo, it means that the moiety contains a carbon atom covalently bonded to at least two hydrogens (e.g., CH₂), and the two hydrogen radicals are substituted with oxo or thioxo to form C═O or C═S, respectively.

The term “alkenyl” means a straight or branched hydrocarbyl chain containing one or more double bonds. Each carbon-carbon double bond may have either cis or trans geometry within the alkenyl moiety, relative to groups substituted on the double bond carbons. Non-limiting examples of alkenyl groups include ethenyl (vinyl), 2-propenyl, 3-propenyl, 1,4-pentadienyl, 1,4-butadienyl, 1-butenyl, 2-butenyl, and 3-butenyl.

The term “alkenylene” refers to a divalent unsaturated hydrocarbyl chain which may be linear or branched and which has at least one carbon-carbon double bond. Non-limiting examples of alkenylene groups include —C(H)═C(H)—, —C(H)═C(H)—CH₂—, —C(H)═C(H)—CH₂—CH₂—, —CH₂—C(H)═C(H)—CH₂—, —C(H)═C(H)—CH(CH₃)—, and —CH₂—C(H)═C(H)—CH(CH₂CH₃)—.

The term “alkyl” means a straight or branched saturated hydrocarbyl chain. Non-limiting examples of alkyl groups include methyl, ethyl, n-propyl, isopropyl, n-butyl, isobutyl, sec-butyl, t-butyl, pentyl, iso-amyl, and hexyl.

The term “alkylene” denotes a divalent saturated hydrocarbyl chain which may be linear or branched. Representative examples of alkylene include, but are not limited to, —CH₂—, —CH₂CH₂—, —CH₂CH₂CH₂—, —CH₂CH₂CH₂CH₂—, and —CH₂CH(CH₃)CH₂—.

The term “alkynyl” means a straight or branched hydrocarbyl chain containing one or more triple bonds. Non-limiting examples of alkynyl include ethynyl, 1-propynyl, 2-propynyl, 3-propynyl, decynyl, 1-butynyl, 2-butynyl, and 3-butynyl.

The term “alkynylene” refers to a divalent unsaturated hydrocarbon group which may be linear or branched and which has at least one carbon-carbon triple bonds. Representative alkynylene groups include, by way of example, —C≡C—, —C≡C—CH₂—, —C≡C—CH₂—CH₂—, —CH₂—C≡C—CH₂—, —C≡C—CH(CH₃)—, and —CH₂—C≡C—CH(CH₂CH₃)—.

The term “carbocycle” or “carbocyclic” or “carbocyclyl” refers to a saturated (e.g., “cycloalkyl”), partially saturated (e.g., “cycloalkenyl” or “cycloalkynyl”) or completely unsaturated (e.g., “aryl”) ring system containing zero heteroatom ring atom. “Ring atoms” or “ring members” are the atoms bound together to form the ring or rings. A carbocycle may be, without limitation, a single ring, two fused rings, or bridged or spiro rings. A substituted carbocycle may have either cis or trans geometry. Representative examples of carbocycle groups include, but are not limited to, cyclopropyl, cyclobutyl, cyclopentyl, cyclohexyl, cycloheptyl, cyclooctyl, cyclopentenyl, cyclopentadienyl, cyclohexadienyl, adamantyl, decahydro-naphthalenyl, octahydro-indenyl, cyclohexenyl, phenyl, naphthyl, indanyl, 1,2,3,4-tetrahydro-naphthyl, indenyl, isoindenyl, decalinyl, and norpinanyl. A carbocycle group can be attached to the parent molecular moiety through any substitutable carbon ring atom. Where a carbocycle group is a divalent moiety linking two other elements in a depicted chemical structure (such as W in Formula II), the carbocycle group can be attached to the two other elements through any two substitutable ring atoms. Likewise, where a carbocycle group is a trivalent moiety linking three other elements in a depicted chemical structure, the carbocycle group can be attached to the three other elements through any three substitutable ring atoms, respectively.

The term “carbocyclylalkyl” refers to a carbocyclyl group appended to the parent molecular moiety through an alkylene group. For instance, C₃-C₆carbocyclylC₁-C₆alkyl refers to a C₃-C₆carbocyclyl group appended to the parent molecular moiety through C₁-C₆alkylene.

The term “cycloalkenyl” refers to a non-aromatic, partially unsaturated carbocyclyl moiety having zero heteroatom ring member. Representative examples of cycloalkenyl groups include, but are not limited to, cyclobutenyl, cyclopentenyl, cyclohexenyl, and octahydronaphthalenyl.

The term “cycloalkyl” refers to a saturated carbocyclyl group containing zero heteroatom ring member. Non-limiting examples of cycloalkyls include cyclopropyl, cyclobutyl, cyclopentyl, cyclohexyl, cycloheptyl, cyclooctyl, decalinyl and norpinanyl.

The prefix “halo” indicates that the substituent to which the prefix is attached is substituted with one or more independently selected halogen radicals. For example, “C₁-C₆haloalkyl” means a C₁-C₆alkyl substituent wherein one or more hydrogen atoms are replaced with independently selected halogen radicals. Non-limiting examples of C₁-C₆haloalkyl include chloromethyl, 1-bromoethyl, fluoromethyl, difluoromethyl, trifluoromethyl, and 1,1,1-trifluoroethyl. It should be recognized that if a substituent is substituted by more than one halogen radical, those halogen radicals may be identical or different (unless otherwise stated).

The term “heterocycle” or “heterocyclo” or “heterocyclyl” refers to a saturated (e.g., “heterocycloalkyl”), partially unsaturated (e.g., “heterocycloalkenyl” or “heterocycloalkynyl”) or completely unsaturated (e.g., “heteroaryl”) ring system where at least one of the ring atoms is a heteroatom (i.e., nitrogen, oxygen or sulfur), with the remaining ring atoms being independently selected from the group consisting of carbon, nitrogen, oxygen and sulfur. A heterocycle may be, without limitation, a single ring, two fused rings, or bridged or spiro rings. A heterocycle group can be linked to the parent molecular moiety via any substitutable carbon or nitrogen atom(s) in the group. Where a heterocycle group is a divalent moiety that links two other elements in a depicted chemical structure (such as W in Formula II), the heterocycle group can be attached to the two other elements through any two substitutable ring atoms. Likewise, where a heterocycle group is a trivalent moiety that links three other elements in a depicted chemical structure, the heterocycle group can be attached to the three other elements through any three substitutable ring atoms, respectively.

A heterocycle may be, without limitation, a monocycle which contains a single ring. Non-limiting examples of monocycles include furanyl, dihydrofuranyl, tetrahydrofuranyl, pyrrolyl, isopyrrolyl, pyrrolinyl, pyrrolidinyl, imidazolyl, isoimidazolyl, imidazolinyl, imidazolidinyl, pyrazolyl, pyrazolinyl, pyrazolidinyl, triazolyl, tetrazolyl, dithiolyl, oxathiolyl, oxazolyl, isoxazolyl, thiazolyl, isothiazolyl, thiazolinyl, isothiazolinyl, thiazolidinyl, isothiazolidinyl, thiodiazolyl, oxathiazolyl, oxadiazolyl (including 1,2,3-oxadiazolyl, 1,2,4-oxadiazolyl (also known as “azoximyl”), 1,2,5-oxadiazolyl (also known as “furazanyl”), and 1,3,4-oxadiazolyl), oxatriazolyl (including 1,2,3,4-oxatriazolyl and 1,2,3,5-oxatriazolyl), dioxazolyl (including 1,2,3-dioxazolyl, 1,2,4-dioxazolyl, 1,3,2-dioxazolyl, and 1,3,4-dioxazolyl), oxathiolanyl, pyranyl (including 1,2-pyranyl and 1,4-pyranyl), dihydropyranyl, pyridinyl, piperidinyl, diazinyl (including pyridazinyl (also known as “1,2-diazinyl”), pyrimidinyl (also known as “1,3-diazinyl”), and pyrazinyl (also known as “1,4-diazinyl”)), piperazinyl, triazinyl (including s-triazinyl (also known as “1,3,5-triazinyl”), as-triazinyl (also known 1,2,4-triazinyl), and v-triazinyl (also known as “1,2,3-triazinyl), oxazinyl (including 1,2,3-oxazinyl, 1,3,2-oxazinyl, 1,3,6-oxazinyl (also known as “pentoxazolyl”), 1,2,6-oxazinyl, and 1,4-oxazinyl), isoxazinyl (including o-isoxazinyl and p-isoxazinyl), oxazolidinyl, isoxazolidinyl, oxathiazinyl (including 1,2,5-oxathiazinyl or 1,2,6-oxathiazinyl), oxadiazinyl (including 1,4,2-oxadiazinyl and 1,3,5,2-oxadiazinyl), morpholinyl, azepinyl, oxepinyl, thiepinyl, thiomorpholinyl, and diazepinyl.

A heterocycle may also be, without limitation, a bicycle containing two fused rings, such as, for example, naphthyridinyl (including [1,8] naphthyridinyl, and [1,6] naphthyridinyl), thiazolpyrimidinyl, thienopyrimidinyl, pyrimidopyrimidinyl, pyridopyrimidinyl, pyrazolopyrimidinyl, indolizinyl, pyrindinyl, pyranopyrrolyl, 4H-quinolizinyl, purinyl, pyridopyridinyl (including pyrido[3,4-b]-pyridinyl, pyrido[3,2-b]-pyridinyl, and pyrido[4,3-b]-pyridinyl), pyridopyrimidine, and pteridinyl. Other non-limiting examples of fused-ring heterocycles include benzo-fused heterocyclyls, such as indolyl, isoindolyl, indoleninyl (also known as “pseudoindolyl”), isoindazolyl (also known as “benzpyrazolyl” or indazolyl), benzazinyl (including quinolinyl (also known as “1-benzazinyl”) and isoquinolinyl (also known as “2-benzazinyl”)), benzimidazolyl, phthalazinyl, quinoxalinyl, benzodiazinyl (including cinnolinyl (also known as “1,2-benzodiazinyl”) and quinazolinyl (also known as “1,3-benzodiazinyl”)), benzopyranyl (including “chromenyl” and “isochromenyl”), benzothiopyranyl (also known as “thiochromenyl”), benzoxazolyl, indoxazinyl (also known as “benzisoxazolyl”), anthranilyl, benzodioxolyl, benzodioxanyl, benzoxadiazolyl, benzofuranyl (also known as “coumaronyl”), isobenzofuranyl, benzothienyl (also known as “benzothiophenyl”, “thionaphthenyl”, and “benzothiofuranyl”), isobenzothienyl (also known as “isobenzothiophenyl”, “isothionaphthenyl”, and “isobenzothiofuranyl”), benzothiazolyl, 4,5,6,7-tetrahydrobenzo[d]thiazolyl, benzothiadiazolyl, benzimidazolyl, benzotriazolyl, benzoxazinyl (including 1,3,2-benzoxazinyl, 1,4,2-benzoxazinyl, 2,3,1-benzoxazinyl, and 3,1,4-benzoxazinyl), benzisoxazinyl (including 1,2-benzisoxazinyl and 1,4-benzisoxazinyl), and tetrahydroisoquinolinyl.

A heterocycle may also be, without limitation, a spiro ring system, such as, for example, 1,4-dioxa-8-azaspiro[4.5]decanyl.

A heterocycle may comprise one or more sulfur atoms as ring members; and in some cases, the sulfur atom(s) is oxidized to SO or SO₂. The nitrogen heteroatom(s) in a heterocyclyl may or may not be quaternized, and may or may not be oxidized to N-oxide. In addition, the nitrogen heteroatom(s) may or may not be N-protected.

The compounds of Formulas I, I′, II and II′ can also be isotopically substituted. Preferred isotopic substitutions include substitutions with stable or nonradioactive isotopes such as deuterium, ¹³C, ¹⁵N or ¹⁸O. Incorporation of a heavy atom, such as substitution of deuterium for hydrogen, can give rise to an isotope effect that could alter the pharmacokinetics of the eventual drug. In one example, at least 5 mol % (e.g., at least 10 mol %) of hydrogen in a compound of Formula I, I′, II or II′ is substituted with deuterium. In another example, at least 25 mol % of hydrogen in a compound of Formula I, I′, II or II′ is substituted with deuterium. In a further example, at least 50, 60, 70, 80 or 90 mol % of hydrogen in a compound of Formula I, I′, II or II′ is substituted with deuterium. The natural abundance of deuterium is about 0.015%. Deuterium substitution or enrichment can be achieved, without limitation, by either exchanging protons with deuterium or by synthesizing the molecule with enriched or substituted starting materials. Other methods known in the art can also be used for isotopic substitutions.

It should be understood that the above-described embodiments and the following example are given by way of illustration, not limitation. Various changes and modifications within the scope of the present invention will become apparent to those skilled in the art from the present description.

### Example

A solution of Compound K and cyclopropylsulfonamide in N-methylpyrrolidinone is dried by azeotropic distillation with isopropyl acetate. The solution is cooled and N,N-carbonyldiimidazole is added while sparging nitrogen through the solution. The reaction reaches greater than 99% conversion to the acyl imidazolide (Scheme III). To a separate vessel is added N,N-carbonyldiimidazole, isopropyl acetate, and diazabicycloundecene. The diazabicycloundecene solution is then transferred to the acyl imidazolide reaction solution. The nitrogen sparge is stopped. The reaction mixture is heated to 40° C. and after stirring for 1-16 hours, the reaction reaches over 99% conversion of the azlactone (Scheme III). Isopropyl acetate is added, followed by 2 M aqueous phosphorice acid. The biphasic mixture is heated to 35° C. and the layers are separated. The organic layer is further diluted with isopropyl acetate and washed twice with 5% aqueous sodium chloride at 35° C. The organic layer is treated with carbon, followed by an isopropyl acetate wash. The organic layer is concentrated to produce a solution of Compound L.

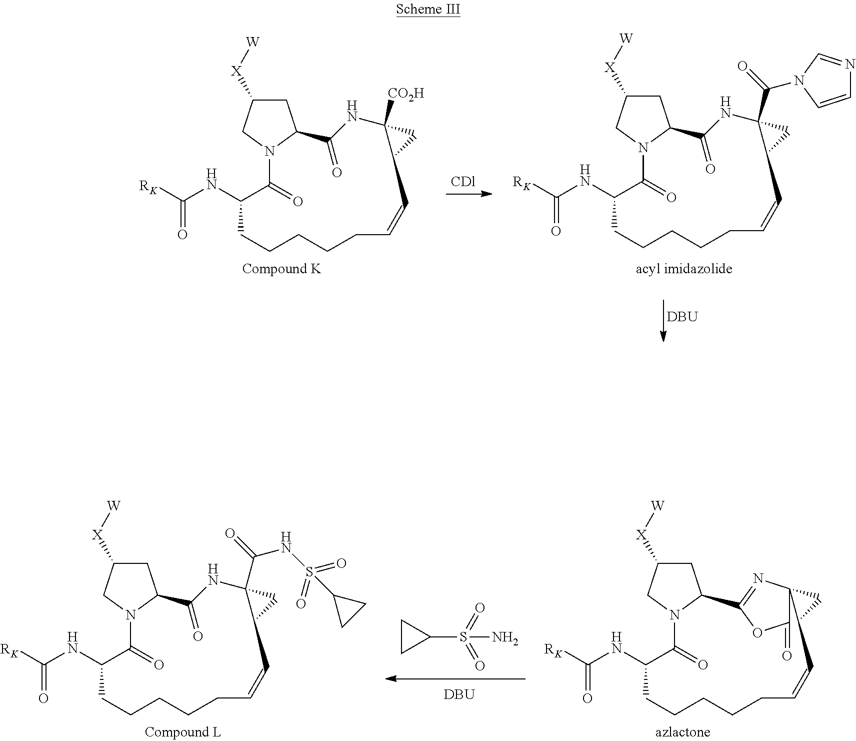

In Compound K, R_{K} is R_{S} or —O—R_{S}, and W, X and R_{S} are as defined above for Formula I. Preferably, X is O, W is an optionally substituted fused bicycle or fused tricycle, and R_{K} is R_{L} or —O—R_{L}, wherein R_{L} is (i) C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle; or (ii) 3- to 6-membered carbocycle or heterocycle, and wherein each 3- to 6-membered carbocycle or heterocycle in R_{L} is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl. Highly preferably, X is O, W is an optionally substituted fused bicycle or fused tricycle, and R_{K} is a 3- to 6-membered carbocycle or heterocycle, which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

Also preferably, X is —OC(O)—, W is an optionally substituted fused bicycle or fused tricycle, and R_{K} is R_{L} or —O—R_{L}, wherein R_{L} is (i) C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle; or (ii) 3- to 6-membered carbocycle or heterocycle, and wherein each 3- to 6-membered carbocycle or heterocycle in R_{L} is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl. Highly preferably, X is −OC(O)—, W is an optionally substituted fused bicycle or fused tricycle, and R_{K} is —O—R_{L}, wherein R_{L} is C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle.

The foregoing description of the present invention provides illustration and description, but is not intended to be exhaustive or to limit the invention to the precise one disclosed. Modifications and variations are possible in light of the above teachings or may be acquired from practice of the invention. Thus, it is noted that the scope of the invention is defined by the claims and their equivalents.

What is claimed is:

1. A compound of Formula I, or a salt thereof, 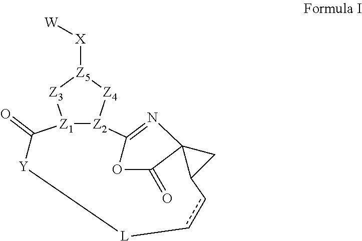
wherein:
W is optionally substituted carbocycle or heterocycle;
X is absent, —O—, —S—, —N(R_{N})—, —OC(O)—, —C(O)—, —C(O)O—, —N(R_{N})C(O)—, —C(O)N(R_{N})—, —S(O)— or —S(O)₂—; or X is optionally substituted C₁-C₆alkylene, C₂-C₆alkenylene or C₂-C₆alkynylene, each of said C₁-C₆alkylene, C₂-C₆alkenylene or C₂-C₆alkynylene containing 0, 1, 2, or 3 heteroatoms independently selected from O, S or N;
Z₁, Z₂ and Z₅ are each independently —C(R_{C})— or —N—;
Z₃ and Z₄, together with Z₁, Z₂ and Z₅, form an optionally substituted 5-membered, 6-membered or 7-membered carbocycle or heterocycle;
Y is C(R_{A}R_{B}) or N(R_{A})
L is optional substituted C₃-C₈alkylene, C₃-C₈alkenylene or C₃-C₈alkynylene, each said C₃-C₈alkylene, C₃-C₈alkenylene or C₃-C₈alkynylene containing 0, 1, 2, or 3 heteroatoms independently selected from O, S or N;
R_{N} is independently selected at each occurrence from hydrogen; or optionally substituted C₁-C₆ alkyl, C₂-C₆alkenyl or C₂-C₆alkynyl; or optionally substituted 3- to 6-membered carbocycle or heterocycle;
R_{C} is independently selected at each occurrence from hydrogen, halogen, hydroxy, mercapto, amino, carboxy, nitro, oxo, phosphonoxy, phosphono, thioxo, formyl or cyano; or optionally substituted C₁-C₆alkyl, C₂-C₆alkenyl or C₂-C₆alkynyl; or optionally substituted 3- to 6-membered carbocycle or heterocycle;
each R_{A} and R_{B} is independently selected at each occurrence from hydrogen, halogen, hydroxy, mercapto, amino, carboxy, nitro, phosphonoxy, phosphono, formyl, cyano, or -L₁-R_{D};
L₁ is independently selected at each occurrence from absent; or optionally substituted C₁-C₆alkylene, C₂-C₆alkenylene or C₂-C₆alkynylene, each of said C₁-C₆alkylene, C₂-C₆alkenylene or C₂-C₆alkynylene containing 0, 1, 2, or 3 heteroatoms independently selected from O, S or N;
R_{D} is independently selected at each occurrence from —O—R_{S}, —S—R_{S}, —C(O)R_{S}, —OC(O)R_{S}, —C(O)OR_{S}, —N(R_{N})R_{S}, —S(O)R_{S}, —SO₂R_{S}, —C(O)N(R_{N})R_{S}, N(R_{N})C(O)R_{S}, —N(R_{N})C(O)N(R_{N})R_{S}, —N(R_{N})SO₂R_{S}, —SO₂N(R_{N})R_{S}, —N(R_{N})SO₂N(R_{N})R_{S}, —N(R_{N})S(O)N(R_{N})R_{S}, —OS(O)—R_{S}, —OS(O)₂—R_{S}, —S(O)₂OR_{S}, —S(O)OR_{S}, —OC(O)OR_{S}, —N(R_{N})C(O)OR_{S}, —OC(O)N(R_{N})R_{S}, —N(R_{N})S(O)—R_{S}, —S(O)N(R_{N})R_{S}, —P(O)(OR_{S})₂, or —C(O)N(R_{N})C(O)—R_{S}; or optionally substituted C₁-C₆alkyl, C₂-C₆alkenyl or C₂-C₆alkynyl; or optionally substituted carbocycle or heterocycle;
R_{S} is independently selected at each occurrence from hydrogen; optionally substituted C₁-C₆alkyl, C₂-C₆alkenyl or C₂-C₆alkynyl; or optionally substituted carbocycle or heterocycle; and [symbol]
denotes optionally substituted —CH₂—CH₂— or optionally substituted —CH═CH—.

2. The compound or salt of claim 1, wherein W is an optionally substituted 9-, 10- or 11-membered carbocycle or heterocycle which comprises two fused rings.

3. The compound or salt of claim 2, wherein Z₁ is N, Z₂ is C(R_{C}), Z₃ is C(R_{C})₂, Z₄ is C(R_{C})₂, and Z₅ is C(R_{C}).

4. The compound or salt of claim 2, wherein Z₁ is N, Z₂ is C(H), Z₃ is CH₂, Z₄ is CH₂, and Z₅ is CH.

5. The compound or salt of claim 1, wherein W is an optionally substituted 12-, 13-, 14-, 15-, or 16-membered carbocycle or heterocycle which comprises three fused rings, provided that W is not

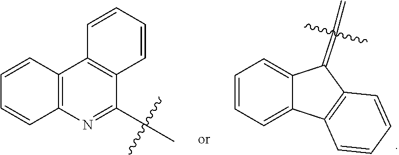 [SMILES: *c1nc2ccccc2c2ccccc12 ; *=C1c2ccccc2-c2ccccc21]

6. The compound or salt of claim 5, wherein Z₁ is N, Z₂ is C(R_{C}), Z₃ is C(R_{C})₂, Z₄ is C(R_{C})₂, and Z₅ is C(R_{C}).

7. The compound or salt of claim 5, wherein Z₁ is N, Z₂ is C(H), Z₃ is CH₂, Z₄ is CH₂, and Z₅ is CH.

8. The compound or salt according to claim 1, wherein X is —O—.

9. The compound or salt according to claim 1, wherein X is —OC(O)— or —C(O)O—.

10. The compound or salt according to claim 1, wherein L is an optionally substituted, straight C₄-C₆alkylene.

11. The compound or salt according to claim 1, wherein L is —CH₂—CH₂—CH₂—CH₂—CH₂—.

12. The compound or salt according to claim 1, wherein Y is —CH(N(R_{N})C(O)R_{S})—, —CH(N(R_{N})C(O)N(R_{N})R_{S})—, or —CH(N(R_{N})C(O)OR_{S})—.

13. The compound or salt according to claim 1, wherein Y is —CH(N(R_{N})C(O)R_{S}′)—, wherein R_{S}′ is (i) hydrogen; (ii) C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle; or (iii) 3- to 6-membered carbocycle or heterocycle, and wherein each 3- to 6-membered carbocycle or heterocycle in R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

14. The compound or salt according to claim 1, wherein Y is —CH(N(R_{N})C(O)OR_{S}′)—, wherein R_{S}′ is (i) hydrogen; (ii) C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle; or (iii) 3- to 6-membered carbocycle or heterocycle, and wherein each 3- to 6-membered carbocycle or heterocycle in R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

15. The compound or salt according to claim 1, wherein [symbol] is —CH═CH—.

16. The compound or salt according to claim 1, wherein [symbol] is —CH₂—CH₂-which is optionally substituted with one or more halogens.

17. The compound or salt according to claim 1, wherein X is —O—, L is —CH₂—CH₂—CH₂—CH₂—CH₂—, Y is —CH(N(R_{N})C(O)R_{S}′)—, and [symbol] is —CH₂—CH₂— which is substituted with one or more halogens or [symbol] is —CH═CH—, wherein R_{S}′ is (i) hydrogen; (ii) C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle; or (iii) 3- to 6-membered carbocycle or heterocycle, and wherein each 3- to 6-membered carbocycle or heterocycle in R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

18. The compound or salt of claim 17, wherein R_{S}′ is 5- to 6-membered carbocycle or heterocycle which is optionally substituted with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

19. The compound or salt according to claim 1, wherein X is —OC(O)— or —C(O)O—, L is —CH₂—CH₂—CH₂—CH₂—CH₂—, Y is —CH(N(R_{N})C(O)OR_{S}′)—, and [symbol] is —CH₂—CH₂— which is substituted with one or more halogens or [symbol] is —CH═CH—, wherein R_{S}′ is (i) hydrogen; (ii) C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle; or (iii) 3- to 6-membered carbocycle or heterocycle, and wherein each 3- to 6-membered carbocycle or heterocycle in R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

20. The compound or salt of claim 19, wherein R_{S}′ is C₁-C₆alkyl, C₂-C₆alkenyl or C₂-C₆alkynyl, each of which is optionally substituted with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle.

21. A reaction solution comprising the compound or salt according to claim 1.

22. A process of making a compound having Formula A, 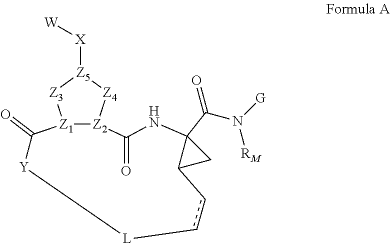
comprising reacting a compound of claim 1 with NH(R_{M})-G, wherein G is —R_{T}, —C(O)R_{T}, —SO₂R_{T}, —S(O)R_{T}, —SO₂N(R_{N})R_{T}, —S(O)N(R_{N})R_{T} or —C(O)OR_{T}, wherein R_{M} is R_{N}, and R_{T} is R_{S}, and wherein W, X, Z₁, Z₂, Z₃, Z₄, Z₅, Y, L, R_{N}, R_{S}, and [symbol] are as defined in claim 1.
